SEQUENCE LISTING (27 sequences)

SEQ ID NO: 1 - DNA, 1609 bp, Rattus norvegicus
gtgtgctggagccactgtcgccgatctcgcgcacgctactgctgctgctcgcccgtcgtcccccatcgtgcactaagcggtcccaaaagattcaaagtccaagatggcagccctcaaggaccagctgattgtgaatcttcttaaggaagaacaggtcccccagaacaagattacagttgttggggttggtgctgttggcatggcttgtgccatcagtatcttaatgaaggacttggctgatgagcttgcccttgttgatgtcatagaagataagctaaagggagagatgatggatcttcagcatggcagccttttccttaagacaccaaaaattgtctccagcaaagattatagtgtgactgcaaactccaagctggtcattatcaccgcgggggcccgtcagcaagagggagagagccggctcaatttggtccagcgaaacgtgaacatcttcaagttcatcattccaaatgttgtgaaatacagtccacagtgcaaactgctcatcgtctcaaacccagtggatatcttgacctacgtggcttggaagatcagcggcttccccaaaaacagagttattggaagtggttgcaatctggattcggctcggttccgttacctgatgggagaaaggctgggagttcatccactgagctgtcacgggtgggtcctgggagagcatggcgactccagtgtgcctgtgtggagtggtgtgaacgtcgccggcgtctccctgaagtctctgaacccgcagctgggcacggatgcagacaaggagcagtggaaggatgtgcacaagcaggtggttgacagtgcatacgaagtgatcaagctgaaaggttacacatcctgggccattggcctctccgtggcagacttggccgagagcataatgaagaaccttaggcgggtgcatcccatttccaccatgattaagggtctctatggaatcaaggaggatgtcttcctcagcgtcccatgtatcctgggacaaaatggaatctcagatgttgtgaaggtgacactgactcctgacgaggaggcccgcctgaagaagagtgcagataccctctggggaatccagaaggagctgcagttctaaagtcttcccagtgtcctagcacttcactgtccaggctgcagcagggtttctatggagaccacgcacttctcatctgagctgtggttagtccagttggtccagttgtgttgaggtggtctgggggaaatctcagttccacagctctaccctgctaagtggtacttgtgtagtggtaacctggttagtgtgacaatcccactgtctccaagacacactgccaactgcatgcaggctttgattaccctgtgagcctgctgcattgctgtgctacgcaccctcaccaaacatgcctaggccatgagttcccagttagttataagctggctccagtgtgtaagtccatcgtgtatatcttgtgcataaatgttctacaggatattttctgtattatatgtgtctgtagtgtacattgcaatattacgtgaaatgtaagatctgcatatggatgatggaaccaaccactcaagtgtcatgccaaggaaaacaccaaataaaccttgaacagtg

SEQ ID NO: 2 - protein, 332 aa, Rattus norvegicus
MAALKDQLIVNLLKEEQVPQNKITVVGVGAVGMACAISILMKDLADELALVDVIEDKLKGEMMDLQHGSLFLKTPKIVSSKDYSVTANSKLVIITAGARQQEGESRLNLVQRNVNIFKFIIPNVVKYSPQCKLLIVSNPVDILTYVAWKISGFPKNRVIGSGCNLDSARFRYLMGERLGVHPLSCHGWVLGEHGDSSVPVWSGVNVAGVSLKSLNPQLGTDADKEQWKDVHKQVVDSAYEVIKLKGYTSWAIGLSVADLAESIMKNLRRVHPISTMIKGLYGIKEDVFLSVPCILGQNGISDVVKVTLTPDEEARLKKSADTLWGIQKELQF

SEQ ID NO: 3 - DNA, 1287 bp, Rattus norvegicus
cgctctacttgctgtaggactctgggtgatgggagaagagcgggagggcagttctttaaccgtgtaagaggagggaccatcccttttggggttcatcaagatgagtaagaactcaggcggctacacatatacggagacctcagtattatttttccatttcaaggtcccaaaagattcaaagtccaagatggcagccctcaaggaccagctgattgtgaatcttcttaaggaagaacaggtcccccagaacaagattacagttgttggggttggtgctgttggcatggcttgtgccatcagtatcttaatgaaggacttggctgatgagcttgcccttgttgatgtcatagaagataagctaaagggagagatgatggatcttcagcatggcagccttttccttaagacaccaaaaattgtctccagcaaagattatagtgtgactgcaaactccaagctggtcattatcaccgcgggggcccgtcagcaagagggagagagccggctcaatttggtccagcgaaacgtgaacatcttcaagttcatcattccaaatgttgtgaaatacagtccacagtgcaaactgctcatcgtctcaaacccagtggatatcttgacctacgtggcttggaagatcagcggcttccccaaaaacaaagttattggaagtggttgcaatctggattcggctcggttccgttacctgatgggagaaaggctgggagttcatccactgagctgtcacgggtgggtcctgggagagcatggcgactccagtgtgcctgtgtggagtggtgtgaacgtcgccggcgtctccctgaagtctctgaacccgcagctgggcacggatgcagacaaggagcagtggaaggatgtgcacaagcaggtggttgacagtgcatacgaagtgatcaagctgaaaggttacacatcctgggccattggcctctccgtggcagacttggccgagagcataatgaagaaccttaggcgggtgcatcccatttccaccatgattaagggtctctatgggatcaaggaggatgtcttcctcagcgtcccatgtatcctgggacaaaatggaatctcagatgttgtgaaggtgacactgactcctgacgaggaggcccgcctgaagaagagtgcagataccctctggggaatccagaaggagctgcagttctaaagtcttcccagtgtcctagcacttcactgtccaggctgcagcagggtttctatggagaccacgcacttctcatctgagctgtggttagtccagttggtcca

SEQ ID NO: 4 - protein, 361 aa, Rattus norvegicus
MSKNSGGYTYTETSVLFFHFKVPKDSKSKMAALKDQLIVNLLKEEQVPQNKITVVGVGAVGMACAISILMKDLADELALVDVIEDKLKGEMMDLQHGSLFLKTPKIVSSKDYSVTANSKLVIITAGARQQEGESRLNLVQRNVNIFKFIIPNVVKYSPQCKLLIVSNPVDILTYVAWKISGFPKNRVIGSGCNLDSARFRYLMGERLGVHPLSCHGWVLGEHGDSSVPVWSGVNVAGVSLKSLNPQLGTDADKEQWKDVHKQVVDSAYEVIKLKGYTSWAIGLSVADLAESIMKNLRRVHPISTMIKGLYGIKEDVFLSVPCILGQNGISDVVKVTLTPDEEARLKKSADTLWGIQKELQF

SEQ ID NO: 5 - protein, 15 aa, Artificial Sequence
  Mitochondrial targeting peptide
MLSRLSLRLLSRYLL

SEQ ID NO: 6 - protein, 34 aa, Zea mays
MYRAAASLASKARQAGSSSAARQVGSRLAWSRNY

SEQ ID NO: 7 - protein, 24 aa, Artificial Sequence
  Mitochondrial targeting peptide
MLSLRQSIRFFPATRTLCSSRYLL

SEQ ID NO: 8 - protein, 27 aa, Artificial Sequence
  Mitochondrial targeting peptide
MLRTSSLFTRRVQPSLFSRNILRLQST

SEQ ID NO: 9 - protein, 17 aa, Artificial Sequence
  Mitochondrial targeting peptide
MLSLRQSIRFFKPATRT

SEQ ID NO: 10 - protein, 21 aa, Artificial Sequence
  Mitochondrial targeting peptide
MFSNLSKRWAQRTLSKSFYST

SEQ ID NO: 11 - protein, 10 aa, Artificial Sequence
  Mitochondrial targeting peptide
MKSFITRNKT

SEQ ID NO: 12 - DNA, 25 bp, Artificial Sequence
  Synthetic oligonucleotide primer
ggaattcgtgtgctggagccactgt

SEQ ID NO: 13 - DNA, 28 bp, Artificial Sequence
  Synthetic oligonucleotide primer
cgcggatcctgtagaacattttatgcac

SEQ ID NO: 14 - DNA, 18 bp, Artificial Sequence
  Synthetic oligonucleotide primer
cgctctacttgctgtagg

SEQ ID NO: 15 - DNA, 22 bp, Artificial Sequence
  Synthetic oligonucleotide primer
gcctggacagtgaagtgctagg

SEQ ID NO: 16 - DNA, 24 bp, Artificial Sequence
  Synthetic oligonucleotide primer
aaccgtgtaagaggagggaccatc

SEQ ID NO: 17 - DNA, 24 bp, Artificial Sequence
  Synthetic oligonucleotide primer
tggaccaactggactaaccacagc

SEQ ID NO: 18 - DNA, 174 bp, Mus musculus
ggagcaacttggcgctctacttgctgtagggctctgggtgatgggagaagagcgggagggcagctttctaaccatataagaggagataccatccccttttggttcatcaagatgagtaagtcctcaggcggctacacgtacacggagacctcggtattatttttccatttcaag

SEQ ID NO: 19 - DNA, 78 bp, Mus musculus
tgcgtgctggagccactgtcgccgagctcggccacgctgcttctcctcgccagtcgcccccccatcgtgcatctagcg

SEQ ID NO: 20 - protein, 29 aa, Mus musculus
MSKSSGGYTYTETSVLFFHFKVSKDSKSK

SEQ ID NO: 21 - protein, 29 aa, Rattus norvegicus
MSKNSGGYTYTETSVLFFHFKVPKDSKSK

SEQ ID NO: 22 - protein, 29 aa, Homo sapiens
MGEPSGGYTYTQTSIFLFHAKIPFGSKSN

SEQ ID NO: 23 - protein, 29 aa, Artificial Sequence
  Consensus leader sequence
MSKXSGGYTYTETSVLFFHFKVPKDSKSK

SEQ ID NO: 24 - DNA, 1680 bp, Mus musculus
gagcaacttggcgctctacttgctgtagggctctgggtgatgggagaagagcgggagggcagctttctaaccatataagaggagataccatccccttttggttcatcaagatgagtaagtcctcaggcggctacacgtacacggagacctcggtattatttttccatttcaaggtctcaaaagattcaaagtccaagatggcaaccctcaaggaccagctgattgtgaatcttcttaaggaagagcaggctccccagaacaagattacagttgttggggttggtgctgttggcatggcttgtgccatcagtatcttaatgaaggacttggcggatgagcttgcccttgttgacgtcatggaagacaaactcaagggcgagatgatggatctccagcatggcagcctcttccttaaaacaccaaaaattgtctccagcaaagactactgtgtaactgcgaactccaagctggtcattatcaccgcgggggcccgtcagcaagagggggagagccggctcaacctggtccagcgaaacgtgaacatcttcaagttcatcattcccaacattgtcaagtacagtccacactgcaagctgctgatcgtctccaatccagtggatatcttgacctacgtggcttggaaaatcagtggctttcccaaaaaccgagtaattggaagtggttgcaatctggattcagcgcggttccgttacctgatgggagagaggctgggggttcacgcgctgagctgtcacggctgggtcctgggagaacatggcgactccagtgtgcctgtgtggagtggtgtgaatgttgccggcgtctccctgaagtctcttaacccagaactgggcactgacgcagacaaggagcagtggaaggaggttcacaagcaggtggtggacagtgcctacgaggtgatcaagctgaaaggttacacatcctgggccattggcctctctgtggcagacttggctgagagcataatgaagaaccttaggcgggtgcatcccatttccaccatgattaagggtctctatggaatcaatgaggatgtcttcctcagtgtcccatgtatcctgggacaaaatggaatctcggatgttgtgaaggtgacactgactcctgaggaagaggcccgcctgaagaagagcgcagacaccctctggggaatccagaaggagctgcagttctaaagtcttccccgtgtcctagcacttcactgtccaggctgcagcagggcttctaggcagaccacacccttctcgtctgagctgtggttagtacagtggtgttgagatggtgtggggaaacatctcactccccacagctctgccctgctgccaagtggtacttgtgtagtggtgacctggttagtgtgacagtcccactgtctctgagacacactgccaactgcaggcttcgattacccctgtgagcctgctgcattgctgccctgcaccaaacagcctaggccgacgagttcccagttaagtcgtataacctggctccagtgtgtacgtccatgatgcatatcttgtgcataaatgttgtacaggatattttatatattatatgtgtctgtagtgtgcattgcaatattatgtgagatgtaagatctgcatatggatgatggaaccaaccacccaagtgtcatgccaaataaaaccttgaacagtg

SEQ ID NO: 25 - protein, 361 aa, Mus musculus
MSKSSGGYTYTETSVLFFHFKVSKDSKSKMATLKDQLIVNLLKEEQAPQNKITVVGVGAVGMACAISILMKDLADELALVDVMEDKLKGEMMDLQHGSLFLKTPKIVSSKDYCVTANSKLVIITAGARQQEGESRLNLVQRNVNIFKFIIPNIVKYSPHCKLLIVSNPVDILTYVAWKISGFPKNRVIGSGCNLDSARFRYLMGERLGVHALSCHGWVLGEHGDSSVPVWSGVNVAGVSLKSLNPELGTDADKEQWKEVHKQVVDSAYEVIKLKGYTSWAIGLSVADLAESIMKNLRRVHPISTMIKGLYGINEDVFLSVPCILGQNGISDVVKVTLTPEEEARLKKSADTLWGIQKELQF

SEQ ID NO: 26 - DNA, 1761 bp, Homo sapiens
ctctggtgtttacttgagaagccctggctgtgtccttgctgtaggagccggagtagctcagagtgatcttgtctgaggaaaggccagccccacttggttaataaaccgcgatgggtgaaccctcaggaggctatacttacacccaaacgtcgatattccttttccacgctaagattccttttggttccaagtccaatatggcaactctaaaggatcagctgatttataatcttctaaaggaagaacagaccccccagaataagattacagttgttggggttggtgctgttggcatggcctgtgccatcagtatcttaatgaaggacttggcagatgaacttgctcttgttgatgtcatcgaagacaaattgaagggagagatgatggatctccaacatggcagccttttccttagaacaccaaagattgtctctggcaaagactataatgtaactgcaaactccaagctggtcattatcacggctggggcacgtcagcaagagggagaaagccgtcttaatttggtccagcgtaacgtgaacatatttaaattcatcattcctaatgttgtaaaatacagcccgaactgcaagttgcttattgtttcaaatccagtggatatcttgacctacgtggcttggaagataagtggttttcccaaaaaccgtgttattggaagtggttgcaatctggattcagcccgattccgttacctgatgggggaaaggctgggagttcacccattaagctgtcatgggtgggtccttggggaacatggagattccagtgtgcctgtatggagtggaatgaatgttgctggtgtctctctgaagactctgcacccagatttagggactgataaagataaggaacagtggaaagaggttcacaagcaggtggttgagagtgcttatgaggtgatcaaactcaaaggctacacatcctgggctattggactctctgtagcagatttggcagagagtataatgaagaatcttaggcgggtgcacccagtttccaccatgattaagggtctttacggaataaaggatgatgtcttccttagtgttccttgcattttgggacagaatggaatctcagaccttgtgaaggtgactctgacttctgaggaagaggcccgtttgaagaagagtgcagatacactttgggggatccaaaaggagctgcaattttaaagtcttctgatgtcatatcatttcactgtctaggctacaacaggattctaggtggaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctagtccaacattttttcccagtgagtcacatcctgggatccagtgtataaatccaatatcatgtcttgtgcataattcttccaaaggatcttattttgtgaactatatcagtagtgtacattaccatataatgtaaaaagatctacatacaaacaatgcaaccaactatccaagtgttataccaactaaaacccccaataaaccttgaacagtg

SEQ ID NO: 27 - protein, 361 aa, Homo sapiens
MGEPSGGYTYTQTSIFLFHAKIPFGSKSNMATLKDQLIYNLLKEEQTPQNKITVVGVGAVGMACAISILMKDLADELALVDVIEDKLKGEMMDLQHGSLFLRTPKIVSGKDYNVTANSKLVIITAGARQQEGESRLNLVQRNVNIFKFIIPNVVKYSPNCKLLIVSNPVDILTYVAWKISGFPKNRVIGSGCNLDSARFRYLMGERLGVHPLSCHGWVLGEHGDSSVPVWSGMNVAGVSLKTLHPDLGTDKDKEQWKEVHKQVVESAYEVIKLKGYTSWAIGLSVADLAESIMKNLRRVHPVSTMIKGLYGIKDDVFLSVPCILGQNGISDLVKVTLTSEEEARLKKSADTLWGIQKELQF

(19) United States
(12) Patent Application Publication
(10) Pub. No.: US 2005/0032160 A1
(43) Pub. Date: Feb. 10, 2005

(54) Cell-based therapies for diabetes mellitus and other glucose intolerant states

(76) Inventors: Christopher B. Newgard, Chapel Hill, NC (US); Mette V. Jensen, Durham, NC (US); A. Dean Sherry, Dallas, TX (US); Shawn C. Burgess, Little Elm, TX (US)

Correspondence Address: Karen A. Magri;Myers Bigel Sibley & Sajovec, P.O. Box 37428, Raleigh, NC, 27627, US

(21) Appl. No.: 10/760,732

(22) Filed: Jan. 21, 2004

Related U.S. Application Data
(60) Provisional application No. 60/441,430, filed on Jan. 21, 2003.

(51) Int. Cl. C07H 21/04; C12N 9/02; C12N 5/08

(52) U.S. Cl. 435/69.1; 435/189; 435/366; 435/320.1; 536/23.2

(57) ABSTRACT

The present invention provides cells comprising an isolated nucleic acid encoding a novel lactate dehydrogenase (LDH) and/or the encoded LDH polypeptide. The invention further provides cells comprising an isolated nucleic acid encoding LDH, wherein the cell is capable of producing and secreting insulin. Also provided are methods of providing fuel-stimulated (e.g., glucose-stimulated) insulin secreting capability to a mammalian subject by implanting cells of the invention (e.g., in a semi-permeable membrane and/or an implantable device) into the subject. Further provided are devices comprising the cells of the invention. In particular embodiments, the device is an implantable device.

### RELATED APPLICATION INFORMATION

This application claims the benefit of U.S. Provisional Application No. 60/441,430, filed Jan. 21, 2003, which is incorporated by reference herein in its entirety.

### GOVERNMENT SUPPORT

The present invention was made, in part, with the support of grant numbers DK 42682 and RR-02584 from the National Institutes of Health. The United States government has certain rights to this invention.

### FIELD OF THE INVENTION

The present invention relates to novel cells comprising isolated nucleic acids encoding lactate dehydrogenase and use of the same for diabetes therapy.

### BACKGROUND OF THE INVENTION

Glucose induced insulin secretion (IS) is tightly coupled to the metabolism of glucose. The far dominant pathway for glucose-derived carbons in pancreatic β-cells is glycolysis. The glycolytic endpoint, pyruvate, is converted to lactate in the cytosol, a process catalyzed by lactate dehydrogenase (LDH); FIG. 1. Only the A form of LDH is believed to be expressed in islets and the activity of the enzyme is reported to be very low (Sekine et al., (1994) J. Biol. Chem. 269:4895). Thus, the vast majority of glucose-derived pyruvate will enter the mitochondria and either be converted into acetyl-CoA for subsequent oxidation in the TCA cycle, or will be carboxylated to oxaloacetate by pyruvate carboxylase. In β-cells, the amount of glucose-derived pyruvate that enters mitochondrial metabolism by carboxylation almost equals that which enters by decarboxylation (MacDonald, (1993) Arch. Biochem. Biophys. 305:205; Khan et al., (1996) J. Biol. Chem. 241:2539). It has been proposed that pyruvate carboxylase-catalyzed anaplerotic influx of pyruvate is linked to an export of NADPH equivalents from the mitochondria which in turn might be important for IS regulation (Macdonald, (1995) J. Biol. Chem. 270:20051). Furthermore, by ¹³C NMR isotopomer analyses, evidence has previously been obtained for a direct correlation between pyruvate cycling (substrate flux through pyruvate carboxylase and back to pyruvate) and IS in the INS-1 derived cell line 832/13 (Lu et al., (2002) Proc; Natl. Acad. Sci. USA 99:2708).

The participation of the pancreatic islets of Langerhans in fuel homeostasis is mediated in large part by their ability to respond to changes in circulating levels of key metabolic fuels by secreting peptide hormones. Insulin secretion from islet β-celis is stimulated by amino acids, three-carbon sugars such as glyceraldehyde, and most prominently, by glucose. The capacity of normal islet β-cells to “sense” a rise in blood glucose concentration, and to respond to elevated levels of glucose (as occurs following ingestion of a carbohydrate-containing meal) by secreting insulin is critical to control of blood glucose levels. Increased insulin secretion in response to a glucose load prevents chronic hyperglycemia in normal individuals by stimulating glucose uptake into peripheral tissues, particularly muscle and adipose tissue.

Mature insulin consists of two polypeptide chains, A and B, joined in a specific manner. However, the initial protein product of the insulin gene in β-cells is not insulin, but preproinsulin. This precursor differs from mature insulin in two ways. Firstly, it has a so-called N-terminal “signal” or “pre” sequence which directs the polypeptide to the rough endoplasmic reticulum, where it is proteolytically processed. The product, proinsulin, still contains an additional connecting peptide between the A and B chains, known as the C-peptide, which permits correct folding of the whole molecule. Proinsulin is then transported to the Golgi apparatus, where enzymatic removal of the C-peptide begins. The processing is completed in the secretory granules, which bud off from the Golgi, travel to, and fuse with, the plasma membrane thus releasing the mature hormone.

Glucose stimulates de novo insulin biosynthesis by increasing transcription, mRNA stability, translation, and protein processing. Glucose also rapidly stimulates the release of pre-stored insulin. While glucose and non-glucose secretagogues can ultimately work through a final common pathway involving alterations in K⁺ and Ca⁺⁺ channel activity and increases in intracellular Ca⁺⁺ (Prentki et al. (1987) Physiol. Rev. 67:1185; Turk et al., (1987) Prog. Lipid Res. 26:125), the biochemical events leading from changes in the levels of a particular fuel to insulin secretion are initially diverse. In the case of glucose, transport into the β-cell and metabolism of this sugar appear to be required for secretion, leading to the hypothesis that its specific stimulatory effect is mediated by, and proportional to, its flux rate through glycolysis and related pathways (Ashcroft, (1980) Diabetologia 18:5, Hedeskov, (1980) Physiol. Rev. 60:442; Meglasson et al., (1986) Diabetes/Metabolism Rev. 2:163; Prentki et al., (1987) Physiol. Rev. 67:1185; Turk et al., (1987) Prog. Lipid Res. 26:125; Malaisse et al., (1990) Biochem Soc. Trans. 18:107). Strong support for this view comes from the finding that non-metabolizable analogs of glucose such as 3-O-methyl or 2-deoxy glucose fail to stimulate insulin release (Ashcroft, (1980) Diabetologia 18:5; Meglasson et al., (1986) Diabetes/Metabolism Rev. 2:163).

There has been a focus by the medical and research communities on the development of new therapeutic approaches for diabetics. Significant effort has been devoted to the strategy of islet or pancreas fragment transplantation as a means for permanent insulin replacement (Lacy et al., (1986) Ann. Rev. Med. 37:33). However, this approach has been hampered by the difficulties associated with obtaining tissue, as well as the finding that transplanted islets are recognized and destroyed by the same autoimmune mechanism responsible for destruction of the patient's original islet β-cells.

Treatment for diabetes is still centered around self-injection of insulin once or twice daily. Both recombinant and non-recombinant methods are currently employed for the industrial production of human insulin for therapeutic use. Recombinant methods generally include the expression of recombinant proinsulin in bacteria or yeast, followed by chemical treatment of the proinsulin to ensure correct disulfide bond linkages between the A and B chains of the mature insulin molecule. The proinsulin produced by microorganisms is processed to insulin by the addition of proteolytic enzymes. Thereafter, the mature insulin peptide must be purified away from the bacterial or yeast proteins, as well as from the added proteases. The bacterial procedure involves 40 distinct steps. Non-recombinant methods typically include the purification of pig insulin from freshly isolated porcine pancreas or pancreatic islets. Each of the above methods suffers from the drawback of being technically difficult and laborious. The latter method is further complicated by the fact that the pancreas is a complex proteinaceous tissue with high levels of active proteases that can degrade insulin, thereby rendering it inactive.

Brooks et al., (1999) Proc Natl. Acad. Sci. USA 96:1129, evaluated LDH isoforms in mitochondria of rat liver and heart tissue by electrophoresis and electron microscopy. These investigators suggested that the mitochondria play a role in cellular lactate oxidation.

Previous reports have indicated that over-expression of LDH impairs IS response (Alcazar et al., (2000) Biochem. Soc. 352:373; Ainscow et al., (2000) Diabetes 49:1149; Zhao et al., (1998) FEBS Lett. 430:213), whereas Ishihara et al., (1999) J. Clin. Invest. 104:1621, found no effect on glucose stimulated IS in INS-1 cells overexpressing LDH. These results may be attributed in part to the fact that the cells employed in these studies do not exhibit a robust glucose-stimulated IS. For example, INS-1 cells are very variable in their performance, depending upon the length of time the cells have been in culture, probably explained by the recent finding that they are comprised of a heterogeneous population of subclones, only some of which are glucose-responsive (Hohmeier et al., (2000) Diabetes 49:424).

Nucleic acid and amino acid sequences encoding LDH have been described (see, e.g., GenBank Accession No. X03753 (mouse; A form); GenBank Accession No. NM_{—}010699 (mouse; A form); Y00309 (mouse; A form); Fukasawa et al., (1987) Genetics 116:99 (mouse; A form); Kayoko et al., (1986) Biochem J. 235: 435 (mouse; A form); Li et al., (1985) Eur. J. Biochem. 149: 215 (mouse; A isoform); Akai et al., (1985) Int. J. Biochem. 17:645 (mouse; A form); GenBank Accession No. NM_{—}017025 (rat; A form); U.S. Pat. No. 6,057,141 (chicken; B form); Hirota et al., (1990) Nucl. Acids Res. 18:6432 (chicken; A form); GenBank Accession No. NM_{—}005566 (human; A form); GenBank Accession No. NM_{—}002300 (human; B form); U.S. Pat. No. 6,503,743 (human); U.S. Pat. No. 6,429,006 and Ishiguro et al., (1991) Gene 91:281 (bovine; A form); GenBank Accession No. AF226154 and U.S. Pat. No. 6,268,189 (Rhizopus oryzae; A form); GenBank Accession No. M22305 (B. megaterium); GenBank Accession No. M19396 (B. stearothermophylus). None of these publications, however, has suggested the presence of a mitochondrial localized form of LDH.

There is a need in the art for improved therapeutic agents for the treatment of diabetes.

### SUMMARY OF THE INVENTION

To gain further insight into the role of mitochondrial metabolism of pyruvate in regulation of insulin secretion (IS) by carbohydrate fuels, a recombinant adenovirus was used to overexpress lactate dehydrogenase A (LDHA) in the β-cell line, 832/13, on the assumption that overexpression of this enzyme would divert pyruvate away from its mitochondrial metabolic pathways. The 832/13 line is a highly differentiated model of β-cell function provided as a subclone of the rat insulinoma cell line INS-1 (Hohmeier et al., (2000) Diabetes 49:424). Surprisingly, the present inventors have discovered that LDH overexpression potentiates insulin secretion (IS) in response to both glucose and pyruvate. The inventors believe that their results are different from those of previous studies in which overexpression of LDH was found to have no effect or to inhibit glucose-stimulated IS because the inventors used the highly glucose-responsive 832/13 cell line. LDH overexpressing cells exhibit increased lactate output, but with no change in glycolytic flux (glucose usage) relative to control cells. In addition, the LDH inhibitor, oxamate, causes a large decrease in lactate production and glucose-stimulated IS. Taken together, these data suggest that LDH overexpression enhances IS despite lesser availability of glucose-derived pyruvate for mitochondrial metabolism, and imply a central role of LDH and lactate in regulation of IS. Interestingly, lactate itself is a poor secretagogue in 832/13 cells relative to glucose or pyruvate, but overexpression of LDH allows lactate to become as effective as the other two fuels.

While not wishing to be bound by any particular theory of the invention, the inventors propose a model involving a compartmentalized form of LDH, including but not limited to an intramitochondrial form. In this model, pyruvate can enter the mitochondria as pyruvate, as lactate, or as two separate pools of pyruvate, the second pool having been produced by the compartmentalized form of LDH. In the absence of LDH overexpression and with lactate as the secretagogue, flux via the first pyruvate pool is insufficient to achieve IS. However, when LDH activity increases, the flux through this pathway is increased. When glucose and pyruvate act as secretagogues, entry via lactate or the second pyruvate pool pathway is limiting but can be enhanced by LDH overexpression. In support of this model, described herein is the cloning of a novel form of LDH and the effects of its overexpression on IS.

Moreover, while not wishing to limit the invention to any particular mechanism of action, the inventors propose that the observed effects of LDH overexpression may be related to the broader effects of pyruvate cycling on IS. ¹³C-isotopomer analysis has previously been applied to a unique set of cell lines derived from rat INS-1 cells which demonstrated a wide range of glucose-stimulated IS from weak to robust (Lu et al., (2002) Proc. Nat. Acad. Sci. USA 99:2708). These investigations found that pyruvate carboxylase-catalyzed pyruvate cycling, but not the fractional contribution of glucose to acetyl-CoA formation (pyruvate dehydrogenase-catalyzed pyruvate metabolism) was correlated with the level of glucose-responsiveness of the various cell lines. Moreover, phenylacetic acid impaired, whereas malate in the form of methyl ester (which is cell-permeable) potentiated, glucose-stimulated IS in the glucose-responsive clones in direct correlation with changes in pyruvate-catalyzed cycling of pyruvate. These results suggested that exchange of pyruvate with TCA cycle intermediates, rather than oxidation of pyruvate via acetyl-CoA, correlates with glucose-stimulated IS.

The current studies indicate that lactate plays an important function in pyruvate cycling and underscore the importance of the mitochondrial pyruvate pool in mediating glucose-stimulated IS. Thus, the present invention points to a broad array of approaches for altering fuel-stimulated IS by impacting pyruvate cycling, cytoplasmic and/or mitochondrial pyruvate pools, lactate and/or pyruvate flux from the cytoplasm into the mitochondria, the conversion of lactate to pyruvate within the mitochondria or at another compartmentalized location, the concentration of NADH in the cytoplasm and, conversely, the concentration of NAD+ in the mitochondria, and the like.

Accordingly, as one aspect, the present invention provides an isolated cell comprising an isolated nucleic acid comprising a nucleotide sequence encoding a mitochondrial lactate dehydrogenase (LDH). In particular embodiments, the cell is an endocrine cell or a secretory cell. The cell can have the capacity to produce insulin and, optionally, can secrete insulin in response to increased concentrations of fuels such as glucose (i.e., demonstrates fuel-sensitive or glucose-sensitive IS) as compared with a comparable cell that does not comprise the isolated nucleic acid encoding LDH.

As a further aspect, the invention provides an isolated cell comprising an isolated nucleic acid comprising a nucleotide sequence encoding LDH, wherein fuel-stimulated IS is enhanced in the cell.

Also provided are pharmaceutical compositions comprising the cells of the invention.

As still another aspect, the invention provides a method of providing fuel-stimulated (e.g., glucose-stimulated) insulin secreting capability to a mammalian subject, comprising implanting into a mammalian subject in need of insulin secreting capability a therapeutically effective amount of a population of cells according to the present invention, optionally positioned in a selectively permeable membrane. In particular embodiments, the subject can be a diabetic subject, a subject with glucose intolerance and/or an obese subject.

As yet a further aspect, the present invention provides a device comprising a population of cells according to the present invention. In particular embodiments, the device is an implantable device.

These and other aspects of the present invention will be discussed in more detail in the description of the invention set forth below.

### BRIEF DESCRIPTION OF THE DRAWINGS

FIG. 1. The metabolic pathways involving pyruvate in pancreatic β-cells. Pyruvate has been shown to have three different destinies in the β-cell. First, pyruvate can be converted to lactate in the cytosol, a process catalyzed by lactate dehydrogenase (LDH). LDH activity is known to be very low in β-cells. Thus, the vast majority of glucose-derived pyruvate will enter the mitochondria and either be converted into acetyl-CoA for subsequent oxidation in the TCA cycle, or will be carboxylated to oxaloacetate by pyruvate carboxylase. In β-cells, the amount of glucose-derived pyruvate that enters mitochondria metabolism by carboxylation almost equals that which enters by decarboxylation. It has been proposed that pyruvate carboxylase-catalyzed anaplerotic influx of pyruvate is linked to an export of NADPH equivalents from the mitochondria which in turn might be important for IS regulation. Furthermore, by ¹³C NMR isotopomer analyses evidence has been obtained for a direct correlation between pyruvate cycling (substrate flux though pyruvate carboxylase and back to pyruvate) and insulin secretion in the INS-1 derived cell line 832/13.

FIG. 2A. Nucleotide sequence for rat LDH_{A} cDNA (Accession Number NM_{—}017025, SEQ ID NO:1).

FIG. 2B. The 332 amino acid sequence of rat LDH_{A} (SEQ ID NO:2). The translation corresponds to nucleotides 104-1609.of SEQ ID NO:1 (FIG. 2A).

FIG. 3. Stimulation of insulin secretion by lactate, pyruvate, and glucose in 832/13 cells. Confluent, insulin secreting 832/13 cells were stimulated for 2 hours with various concentrations of secretagogues as indicated in the figure, and IS was determined by radioimmunoassay of the cell supernatant.

FIG. 4. The effect of rat LDH_{A} overexpression on glucose, pyruvate, and lactate-stimulated insulin secretion in 832/13 cells. 832/13 cells were treated with adenovirus expressing rat LDH_{A} (AdLDH) and beta-galactosidase (Adbetagal), respectively. The LDH activity in cell extracts was monitored by the decrease in absorbance at 340 nm (insert) and the insulin response to glucose, pyruvate, and lactate was examined by radioimmunoassay.

FIG. 5. The effect of rat LDH_{A} overexpression on glucose usage in 832/13 cells. In order to compare glycolytic fluxin cells overexpressing rat LDH_{A} and beta-galactosidase, respectively, cells were incubated for 2 hours in the presence of [3-³H]glucose. After deproteination, supernatants were transferred to microcentrifuge tubes and placed overnight at 50° C. in tightly closed scintillation vials. Glucose usage was determined from the level of ³H₂O released to the scintillation tubes.

FIG. 6. The effect of rat LDH_{A} overexpression on lactate production in 832/13 cells. Lactate production from insulin secreting 832/13 cells treated with AdLDH was compared with output from Adbetagal virus treated control cells using a lactate oxidase/peroxidase linked assay combined with A_{540 nm} measurement. The increased lactate production in LDH overexpressing cells indicates that the overexpressed enzyme functions in the direction of lactate production.

FIG. 7A. The effect of the LDH inhibitor oxamate on lactate output in 832/13 cells. Cells were incubated with ¹³C labeled glucose for 4 hours in the presence or absence of oxamate.

FIG. 7B. The effect of the LDH inhibitor oxamate on insulin secretion in 832/13 cells. Cells were incubated with ¹³C labeled glucose for 4 hours in the presence or absence of oxamate.

FIG. 7C. The effect of the LDH inhibitor oxamate on pyruvate cycling in 832/13 cells. Cells were incubated with ¹³C labeled glucose for 4 hours in the presence or absence of oxamate and pyruvate cycling was determined by ¹³C NMR analysis as described in Lu et al., (2000) Proc. Natl. Acad. Sci. USA 99:2708.

FIG. 7D. The effect of the LDH inhibitor oxamate on insulin secretion in islet β-cells. Cells were incubated with ¹³C labeled glucose for 4 hours in the presence or absence of oxamate.

FIG. 8 depicts a model involving a mitochondrial form of LDH_{A}. Pyruvate can enter the mitochondria in two ways, either as pyruvate, or as lactate which in turn is converted to pyruvate inside the mitochondria. It is proposed that both these entry pathways need to be active in order to obtain IS.

FIG. 9 depicts the effect of glycerol kinase overexpression in 832/13 cells on insulin excretion, lactate output and pyruvate cycling.

FIG. 10. The organization of the mouse cytoplasmic LDH_{A} gene. The gene (12.9 kb) for cytoplasmic LDH_{A} consists of 8 exons (in black) with the translational start site present in the second exon that gives rise to a protein of 332 amino acids. An alternative exon (diagonally striped, SEQ ID NO:18) between exon 1 (SEQ ID NO:19) and exon 2 has been identified and contains an alternative start site (capital letters). Transcripts which have this alternative exon spliced to the 5′ end of exon 2 will give rise to a LDH protein with an additional 29 amino acids (SEQ ID NO:20) at the N-terminal end that have the features of a mitochondrial targeting signal.

FIG. 11 shows the peptide sequence of the 29 amino acid leader sequence of the putative mit-LDH_{A} from mouse (SEQ ID NO:20), rat (SEQ ID NO:21) and human (SEQ ID NO:22), along with the consensus peptide sequence (SEQ ID NO:23).

FIG. 12A shows the nucleic acid sequence (SEQ ID NO:3) for the cDNA clone of the rat mitochondrial form of LDH_{A}.

FIG. 12B shows the amino acid sequence (SEQ ID NO:4) for the rat mitochondrial form of LDH_{A} from the translation of nucleotides 101-1186 of SEQ ID NO:3.

FIG. 12C shows the nucleic acid sequence (SEQ ID NO:24) for the cDNA clone of the mouse mitochondrial form of LDH_{A}.

FIG. 12D shows the amino acid sequence (SEQ ID NO:25) for the mouse mitochondrial form of LDH_{A} from the translation of nucleotides 111-1193 of SEQ ID NO:24.

FIG. 12E shows the nucleic acid sequence (SEQ ID NO:26) for the cDNA clone of the human mitochondrial form of LDH_{A}.

FIG. 12F shows the amino acid sequence (SEQ ID NO:27) for the human mitochondrial form of LDH_{A} from the translation of nucleotides 111-1193 of SEQ ID NO:26.

FIGS. 13A-D shows the alignment of rat (SEQ ID NO:3), mouse (SEQ ID NO:24), and human (SEQ ID NO:26) mitochondrial LDH_{A} cDNA sequences.

FIG. 14. The effect of mitLDH_{A} and LDH_{A} on fuel-mediated IS in the 832/13 cells. The inset depicts the activity of LDH for each experiment.

### DETAILED DESCRIPTION OF THE PREFERRED EMBODIMENTS

The present invention will now be described with reference to the accompanying drawings, in which preferred embodiments of the invention are shown. This invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Except as otherwise indicated, standard methods can be used for the production of viral and non-viral vectors, manipulation of nucleic acid sequences, production of transformed cells, and the like according to the present invention. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

I. Definitions.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

The term “modulate,” “modulates” or “modulation” refers to enhancement (e.g., an increase) or inhibition (e.g., a reduction) in the specified activity.

Similarly, the term “modulate LDH activity,” “modulates LDH activity” or “modulation of LDH activity” refers to an enhancement (e.g., an increase) or inhibition (e.g., a reduction) in LDH enzymatic activity (for example, as measured by conversation of pyruvate to lactate). In representative embodiments, modulation of LDH activity within the cells of the pancreatic islets of Langerhans is of interest (e.g., in the islet β-cells). Those skilled in the art will appreciate that in the practice of particular embodiments of the invention, it will not be possible or will be difficult to measure LDH activity within the pancreatic islets of a live animal. Thus, according to the invention, a determination as to whether a compound modulates LDH activity can be made in vivo in an intact animal or a tissue removed from the animal; alternatively, modulation of LDH enzymatic activity can be detected in isolated pancreatic islet β-cells, cell lines derived from pancreatic islet β-cells (e.g., insulinoma cells), or any other suitable cell in culture.

The term “overexpress,” “overexpresses” or “overexpression” as used herein in connection with an isolated nucleic acid encoding a polypeptide refers to expression that results in higher levels of polypeptide production than exist in the cell in its native (untransformed) state.

The term “overexpress,” “overexpresses” or “overexpression” as used herein in connection with isolated nucleic acids encoding an LDH transgene refers to expression that results in higher levels of LDH polypeptide than exist in the cell in its native (untransformed) state. Overexpression of LDH can result in levels that are 25%, 50%, 100%, 200%, 500%, 1000%, 2000% or higher in the cell. Further, the LDH can be introduced into a cell that does not produce the specified form of LDH (e.g., A isoform or mitochondrial LDH) encoded by the transgene or does so only at negligible levels.

As used herein, the term “diabetes” is used interchangeably with the term “diabetes mellitus.” The terms “diabetes” and “diabetes mellitus” are intended to encompass both insulin dependent and non-insulin dependent (Type I and Type II, respectively) diabetes mellitus, unless one condition or the other is specifically indicated.

The term “insulin secretion” (IS) as used herein refers to secretion of insulin from a cell, e;g., into the systemic circulation or cell culture medium, and will typically refer to secretion of insulin from pancreatic islet β-cells, although it can also refer to secretion from cells which have been engineered to express a recombinant insulin (for example, artificial β-cells). Insulin secretion can be assessed directly, for example, by measuring plasma insulin concentrations using art-known methods such as radioimmunoassay. Alternatively, insulin secretion can be indirectly evaluated by measuring, for example, changes in plasma glucose concentrations (or glucose concentrations in cell culture medium).

“Fuel-stimulated insulin secretion” refers to the commencement or enhancement of insulin secretion in response to an elevation in the extracellular concentration of carbohydrates (e.g., glucose, glycerol, glyceraldehyde), amino acids and/or fatty acids. In particular embodiments, fuel-stimulated insulin secretion results in an increase of at least about 2-fold, 3-fold, 4-fold, 5-fold, 6-fold, 8-fold, 10-fold, twelve-fold, or even fifteen-fold or more in insulin secretion over baseline levels in the presence of a sufficiently high concentration of fuels; In other words, the level of fuel-stimulated insulin secretion can be dependent on the concentration of the fuel(s), but the maximal elevation in insulin secretion is at least about 2-fold, 3-fold, 4-fold, 5-fold, 6-fold, 8-fold, 10-fold, twelve-fold, or even fifteen-fold or more over baseline levels. Alternatively, fuel-stimulated insulin secretion can be commenced in a cell or subject that did not previously have any detectable fuel-stimulated insulin secretion (or only negligible levels).

“Glucose-stimulated insulin secretion” refers to the compensatory secretion of insulin in response to an elevation in serum glucose (e.g., following a meal or a glucose challenge) or, in the case of cultured cells, to an elevation of glucose concentration in the cell culture medium. In particular embodiments, glucose-stimulated insulin secretion results in an increase of at least about 2-fold, 3-fold, 4-fold, 5-fold, 6-fold, 8-fold, 10-fold, twelve-fold, or even fifteen-fold or more in insulin secretion over baseline levels in the presence of a sufficiently high concentration of glucose. In other words, the level of glucose-stimulated insulin secretion can be dependent on the glucose concentration, but the maximal elevation in insulin secretion is at least about 2-fold, 3-fold, 4-fold, 5-fold, 6-fold, 8-fold, 10-fold, twelve-fold or even fifteen-fold or more over baseline levels. Alternatively, glucose-stimulated insulin secretion can be commenced in a cell or subject that did not previously have any detectable glucose-stimulated insulin secretion (or only negligible levels).

The term “enhance,” “enhances,” “enhancing” or “enhancement” with respect to insulin secretion refers to an increase in insulin secretion (e.g., at least about a 1.5-fold, 2-fold, 3-fold, 4-fold, 5-fold, 6-fold, 8-fold, 10-fold, twelve-fold, or even fifteen-fold or more increase), for example, in response to elevated glucose concentrations. Alternatively, these terms can refer to commencing insulin secretion in a cell or subject that did not previously have any detectable insulin secretion. In particular embodiments, fuel-stimulated IS is enhanced in a cell or subject comprising an isolated nucleic acid encoding LDH according to the invention as compared with the level of fuel-stimulated IS in a comparable cell in the absence of the isolated nucleic acid overexpressing LDH.

By “providing fuel-simulated [or glucose-stimulated] insulin secreting capability” to a subject, it is meant that fuel-stimulated (or glucose-stimulated) insulin secreting capability is enhanced as described above. Thus, fuel-stimulated (or glucose-stimulated) insulin secretion can be commenced in a subject that did not previously have detectable fuel-stimulated (or glucose-stimulated) insulin secretion or can be increased above previous levels.

The term “glucose tolerance” refers to a state in which there is proper functioning of the homeostatic mechanisms by which insulin is secreted in response to an elevation in serum glucose concentrations. Impairment in this system results in transient hyperglycemia as the organism is unable to maintain normoglycemia following a glucose load (for example, a carbohydrate containing meal) because of insufficient secretion of insulin from the islet β-cells or because of insensitivity of target tissues to circulating insulin.

“An improvement in glucose tolerance” is a level of amelioration in glucose tolerance that provides some clinical benefit to the subject. Glucose tolerance can be assessed by methods known in the art, such as for example, the oral glucose tolerance test which monitors serum glucose concentrations following an oral glucose challenge. In particular embodiments, an “improvement in glucose tolerance” can result in normalization of fasting or baseline serum glucose concentrations, a reduction in maximal serum glucose concentrations, and/or an improved temporal response to a glucose challenge.

A “transgenic” non-human animal is a non-human animal that comprises a foreign nucleic acid incorporated into the genetic makeup of the animal, such as for example, by stable integration into the genome or by stable maintenance of an episome (e.g., derived from EBV).

A “therapeutically effective” amount as used herein is an amount that provides some improvement or benefit to the subject. Alternatively stated, a “therapeutically-effective” amount is an amount that provides some alleviation, mitigation, or decrease in at least one clinical symptom of glucose intolerance or diabetes in the subject (e.g., improved glucose tolerance, enhanced glucose-stimulated insulin secretion, and the like) as is well-known in the art. Those skilled in the art will appreciate that the therapeutic effects need not be complete or curative, as long as some benefit is provided to the subject.

By the terms “treating” or “treatment of,” it is intended that the severity of the patient's condition is reduced or at least partially improved or modified and that some alleviation, mitigation or decrease in at least one clinical symptom is achieved.

As used herein, a “vector” or “delivery vector” can be a viral or non-viral vector that is used to deliver a nucleic acid to a cell, tissue or subject.

A “recombinant” vector or delivery vector refers to a viral or non-viral vector that comprises one or more heterologous nucleotide sequences (i.e., transgenes), e.g., two, three, four, five or more heterologous nucleotide sequences. Generally, the recombinant vectors of the invention encode LDH, but can also comprise one or more additional heterologous sequences.

As used herein, the term “viral vector” or “viral delivery vector” can refer to a virus particle that functions as a nucleic acid delivery vehicle, and which comprises the vector genome packaged within a virion. Alternatively, these terms can be used to refer to the vector genome when used as a nucleic acid delivery vehicle in the absence of the virion.

A viral “vector genome” refers to the viral genomic DNA or RNA, in either its naturally occurring or modified form. A “recombinant vector genome” is a viral genome (e.g., vDNA) that comprises one or more heterologous nucleotide sequence(s).

A “heterologous nucleotide sequence” will typically be a sequence that is not naturally-occurring in the vector. Alternatively, a heterologous nucleotide sequence can refer to a sequence that is placed into a non-naturally occurring environment (e.g., by association with a promoter with which it is not naturally associated).

By “infectious,” as used herein, it is meant that a virus can enter a cell by natural transduction mechanisms and express viral genes and/or nucleic acids (including transgenes). Alternatively, an “infectious” virus is one that can enter the cell by other mechanisms and express the coding sequences carried within the viral genome therein. As one illustrative example, the vector can enter a target cell by expressing a ligand or binding protein for a cell-surface receptor in the virion or by using an antibody(ies) directed against molecules on the cell-surface followed by internalization of the complex.

As used herein, the term “polypeptide” encompasses both peptides and proteins, unless indicated otherwise.

A “fusion polypeptide” is a polypeptide produced when two heterologous nucleotide sequences or fragments thereof coding for two (or more) different polypeptides not found fused together in nature are fused together in the correct translational reading frame. Illustrative fusion polypeptides include fusions of LDH (or a portion thereof) to all or a portion of glutathione-S-transferase, maltose-binding protein, or a reporter protein (e.g., Green Fluorescent Protein, β-glucuronidase, β-galactosidase, luciferase, etc.).

As used herein, a “functional” polypeptide is one that retains at least one biological activity normally associated with that polypeptide. Preferably, a “functional” polypeptide retains all of the activities possessed by the unmodified peptide. By “retains” biological activity, it is meant that the polypeptide retains at least about 50%, 60%, 75%, 85%, 90%, 95%, 97%, 98%, 99%, or more, of the biological activity of the native polypeptide (and can even have a higher level of activity than the native polypeptide). A “non-functional” polypeptide is one that exhibits essentially no detectable biological activity normally associated with the polypeptide (e.g., at most, only an insignificant amount, e.g., less than about 10% or even 5%).

A “recombinant” nucleic acid is one that has been created using genetic engineering techniques.

A “recombinant polypeptide” is one that is produced from a recombinant nucleic acid.

As used herein, an “isolated” nucleic acid (e.g., an “isolated DNA” or an “isolated vector genome”) means a nucleic acid separated or substantially free from at least some of the other components of the naturally occurring organism or virus, such as for example, the cell or viral structural components or other polypeptides or nucleic acids commonly found associated with the nucleic acid.

Likewise, an “isolated” polypeptide means a polypeptide that is separated or-substantially free from at least some of the-other components of the naturally occurring organism or virus, for example, the cell or viral structural components or other polypeptides or nucleic acids commonly found associated with the polypeptide. As used herein, the “isolated” polypeptide is at least about 25%, 50%, 60%, 70%, 75%, 80%, 85%, 90%, 95%, 97%, 98%, 99% or more pure (w/w).

As used herein an “isolated” cell is a cell that is free or substantially free from at least some of the other components of the naturally occurring organism. An “isolated” cell can be a cultured cell. Alternatively, an “isolated” cell can be a cell in a pharmaceutical composition, positioned in a selectively permeable membrane and/or in an implantable device as described herein. Further, an “isolated” cell can be a cell that has been implanted into a recipient host, e.g., in a pharmaceutical composition, a selectively permeable membrane and/or an implantable device. According to this embodiment, the cell can be derived from the host subject or can be foreign to the subject.

By the term “express,” “expresses” or “expression” of a nucleic acid coding sequence, in particular a LDH coding sequence, it is meant that the sequence is transcribed, and optionally, translated. Typically, according to the present invention, transcription and translation of the coding sequence will result in production of LDH polypeptide.

II. Lactate Dehydrogenase.

Mammalian LDH is a tetrameric enzyme composed of A and B subunits, also known as M (muscle) and H (heart) forms, respectively. There are five isozymes of LDH enzyme resulting from the assembly of homotetramers (AAM or BBBB) or heterotetramers (ABBB, AABB, AAAB) of the A and B isoforms. The B isoform predominates in heart muscle and facilitates the aerobic oxidation of pyruvate. The A subunit predominates in skeletal muscle and liver and is primarily implicated with anaerobic metabolism and pyruvate reduction to lactate. To date, only the A form has been identified in islet β-cells. Another LDH isoform, LDHc, has been identified in spermatozoa.

LDH shares structural similarities with other NAD-binding enzymes. The polypeptide chain of each subunit folds into two clearly separated domains. The two domains have different functions, and appear to each comprise a separate module. One of the domains (domain 1) binds to the coenzyme, NAD/NADH, and the second (domain 2) binds the substrate (e.g., pyruvate or lactate) and also provides the amino acid residues that are involved in catalysis. The coenzyme-binding domain is in the amino-terminal portion of the polypeptide. The active site of the enzyme is the cleft or “vacuole” that is formed between the two domains. The coenzyme-binding site on the one domain and the substrate-binding site on the other are oriented so that the C4 position of the nicotinamide ring is in close proximity to the hydrogen atom to be transferred between the substrate and coenzyme (Introduction to Protein Structure. 1991. Branden and Tooze (eds). Garland Publishing, Inc., New York. pg. 144).

The LDH inhibitor, oxamate, binds at the active site of the enzyme with the NADH coenzyme. Structural analysis of the LDH_{A}-oxamate-NADH complex indicates that NADH binds at the top end of the central parallel beta sheet and the mobile active site loop above it in domain 1. Domain 2 forms the other side of the active site, around and behind the oxamate. There is also an amino-terminal tail that is non-compact in the monomer but wraps around another subunit in the intact tetramer. The active site cleft includes Asn 140, with its side chain nitrogen forming a hydrogen bond with oxygens on both the oxamate and the nicotinamide ring.

The mobile active site loop (residues 94-120) forms as a result of charge changes in the vacuole. It is believed that the amino acid residues Arg 109, Arg 171, His 195 and Asn 140 are important for enzymatic activity. Cortes et al., (1992) Protein Sci 1:892, have reported that formation of the active site vacuole is dependent on the ionizing groups within the vacuole having the same total overall charge as is present in the wild type enzyme complex with NAD+ and lactate (incorporated by reference herein in its entirety for its teachings of the effects of amino acid substitutions and charge on LDH enzymatic activity). Substitution of an Asp residue for Asn 140 resulted in a 10-fold increase in the Km for pyruvate for each unit increase in pH over pH 4.5 up to pH 9. These investigators concluded that the anion of Asp 140 was completely inactive and that it bound pyruvate with a Km that is more than 1,000 times greater than the Km of the neutral protonated Asp 140. Further, this report indicates that the active site vacuole is only sufficiently large to accept substrates up to C4 in the presence of bound coenzyme.

The term “lactate dehydrogenase” or “LDH” as used herein, is intended to be construed broadly and encompasses the A, B and C forms of LDH as well as cytoplasmic, mitochondrial, or otherwise compartmentalized forms of the enzyme (e.g., in the endoplasmic reticulum or membrane-bound within the. cytoplasm). The term “lactate dehydrogenase” or “LDH” also includes modified (e.g., mutated) LDH that retain biological function (i.e., have at least one biological activity of the native LDH protein, e.g., converting pyruvate →lactate), functional -LDH fragments including truncated molecules, and functional LDH fusion polypeptides (e.g., an LDH-maltose binding protein fusion).

Generally, the functional LDH fragment forms an active site cleft and is able to bind substrate (e.g., pyruvate or lactate) and coenzyme (e.g., NAD or NADH). In representative embodiments, a functional LDH fragment comprises at least about 25, 50,100,150 or 200 amino acids of the full-length polypeptide. In other embodiments, the functional LDH fragment comprises domain 1 (coenzyme binding domain) and/or domain 2 (catalytic and substrate binding domain). In still other embodiments, the functional LDH fragment comprises the residues that form the mobile active site loop (amino acids 94-120).

Moreover, the term “lactate dehydrogenase” or “LDH” refers to a single subunit (e.g., A isoform) or a multimer of subunits (e.g., the mature tetrameric enzyme), or both, depending upon the context. In particular embodiments, the LDH is an LDH_{A} monomer or multimeric assembly of LDH_{A} (e.g., a homotetramer).

In illustrative embodiments, the LDH is compartmentalized. For example, the LDH can be a mitochondrial targeted LDH (e.g., to the mitochondrial matrix). By “mitochondria targeted” it is intended that intracellular processing results in a substantial portion of the nascent protein being directed to and localized in the mitochondria (for example, in the mitochondrial matrix, inner membrane and/or intermembrane space). This definition does not exclude the possibility that some or even all of the mitochondria targeted LDH is translocated or leaked into the cytoplasm where it can exert its cellular effects on IS as described herein.

The mitochondria targeted LDH can be naturally occurring or can be produced by recombinant nucleic acid techniques by engineering an LDH polypeptide to be targeted to the mitochondria (e.g., to the mitochondrial matrix) by in-frame fusion of a sequence encoding a mitochondria signal peptide (e.g., of about 10 to about 50 or 100 amino acids or more in length), typically at the amino terminus, as known in the art. Suitable signal sequences for localizing LDH to a mitochondrial compartment of interest can be derived from the precursors of proteins that normally reside in that mitochondrial compartment. Known mitochondrial targeting sequences vary in length, from about-10-70 residues, and the most common sequence similarity among them is the predominance, all along their length, of basic residues, hydroxyl-containing Ser and Thr residues, and small hydrophobic residues (Proteins: Structures and Molecular Properties. Second edition. (1993) Thomas E. Creighton (ed.), W. H. Freeman and Company, New York;

incorporated herein by reference in its entirety for teachings of mitochondrial targeting sequences).

Exemplary mitochondrial targeting peptides (in particular, targeting peptides that result in localization in the mitochondrial matrix) have been described in U.S. Patent Application Publication No. 20020151014 (MLSRLSLRLLSRYLL; SEQ ID NO:5); Whelan and Glaser, (1997) Plant Mol. Biol. 33:771-789 (providing a review of mitochondrial targeting sequences); Close, Pamela S., Cloning and Molecular Characterization of Two Nuclear Genes for Zea mays Mitochondrial Chaperonin 60, Doctoral Thesis, Iowa State University (1993) (describing a mitochondrial signal sequence from the maize chaperonin 60 gene; MYRAAASLASKARQAGSSSAARQVGSRLAWSRNY; SEQ ID NO:6). Other mitochondria targeting sequences that have been reported include the sequence MLSLRQSIRFFPATRTLCSSRYLL (SEQ ID NO:7) and the mitochondria targeting sequences discussed in Proteins: Structures and Molecular Properties. Second edition. (1993) Thomas E. Creighton (ed.), W.H. Freeman and Company, New York. pg. 72 (for example, MLRTSSLFTRRVQPSLFSRNILRLQST, SEQ ID NO:8; MLSLRQSIRFFKPATRT, SEQ ID NO:9; MFSNLSKRWAQRTLSKSFYST (SEQ ID NO:10;-MKSFITRNKT, SEQ ID NO:11).

Alternatively, the mitochondrial targeting sequence can be wholly or partially synthetic.

In still other embodiments, the LDH is a cytoplasmic LDH. By “cytoplasmic” LDH, it is intended that intracellular processing results in a substantial portion of the newly-synthesized LDH protein being directed to and localized in the cytoplasm of the cell.

In still other embodiments, overexpression of the LDH results in enhancement of mitochondrial(pyruvate concentrations.

Any LDH polypeptide or LDH-encoding nucleic acid known in the art can be used according to the present invention. The LDH polypeptide or LDH-encoding nucleic acid can be derived from bacterial, yeast, fungal, plant or animal (e.g., insect, avian (e.g., chicken), mammalian (e.g., rat, mouse, bovine, porcine, ovine, caprine, equine, feline, canine, lagomorph, simian, human and the like) sources.

Exemplary LDH polypeptides and LDH-encoding nucleic acids, include but are not limited to, those disclosed in: GenBank Accession No. X03753 (mouse; A form); GenBank Accession No. NM_{—}010699 (mouse; A form); Y00309 (mouse; A form); Fukasawa et al:, (1987) Genetics 116:99 (mouse; A form); Kayoko et al., (1986) Biochem J. 235: 435 (mouse; A form); Li et al., (1985) Eur. J. Biochem. 149: 215 (mouse; A isoform); Akai et al., (1985) Int. J. Biochem. 17:645 (mouse; A form); GenBank Accession No. NM_{—}017025 (rat; A form); U.S. Pat. No. 6,057,141 (chicken; B form); Hirota et al., (1990) Nucl. Acids Res. 18:6432 (chicken; A form); GenBank Accession No. NM_{—}005566 (human; A form); GenBank Accession No. NM_{—}002300 (human; B form); U.S. Pat. No. 6,503,743 (human); U.S. Pat. No. 6,429,006 and Ishiguro et al., (1991) Gene 91:281 (bovine; A form); GenBank Accession No. AF226154 and U.S. Pat. No. 6,268,189 (Rhizopus oryzae; A form); GenBank Accession No. M22305 (B. megaterium); GenBank Accession No. M19396 (B. stearothermophylus); the disclosures of which are incorporated herein by reference in their entireties for their teachings of LDH coding sequences and proteins.

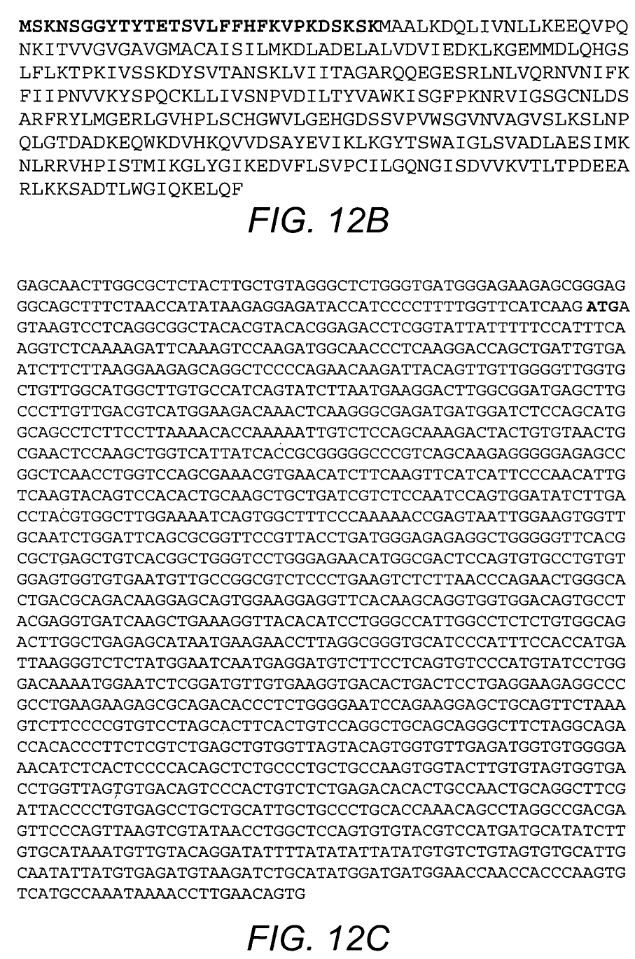

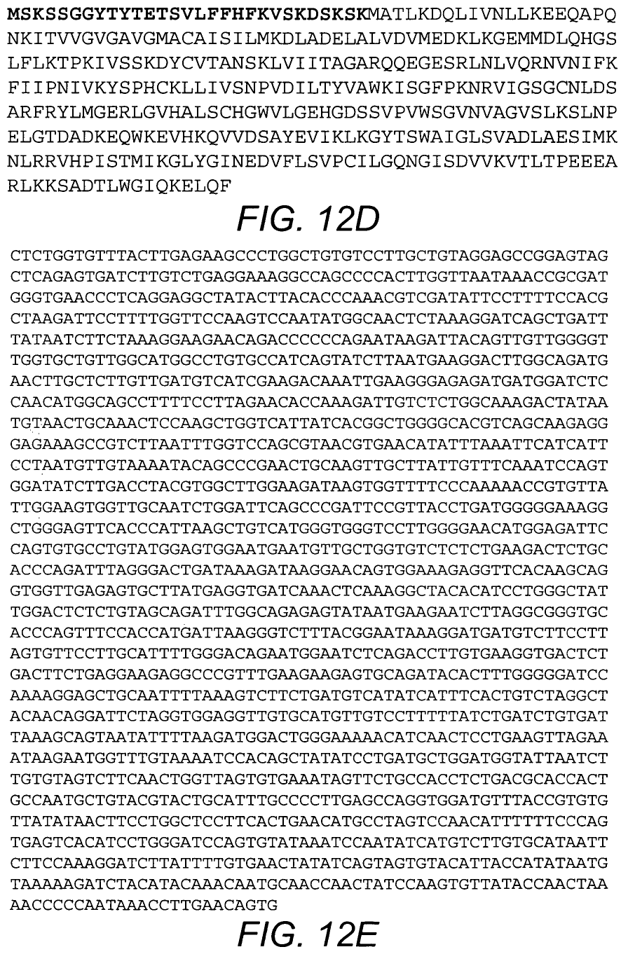

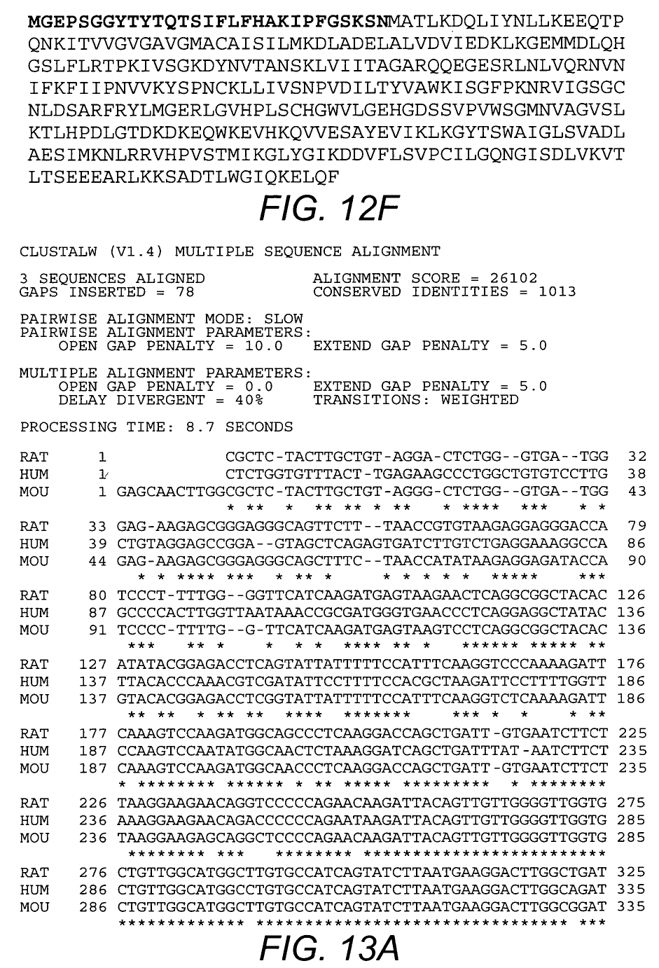

Representative cDNA and amino acid sequences of a rat mitochondrial (or otherwise compartmentalized) LDH_{A} are shown in SEQ ID NO:3 and SEQ ID NO:4, respectively (FIGS. 12A and 12B). Exemplary nucleotide and amino acid sequences of the mitochondrial LDH_{A} from mouse (SEQ ID NO:24 and SEQ ID NO:25, respectively) and human mitochondrial LDH_{A} (SEQ ID NO:26 and SEQ ID NO:27, respectively) are also disclosed (FIGS. 12C-12F). Other mitochondrial LDH encompassed by the present invention are described in more detail below.

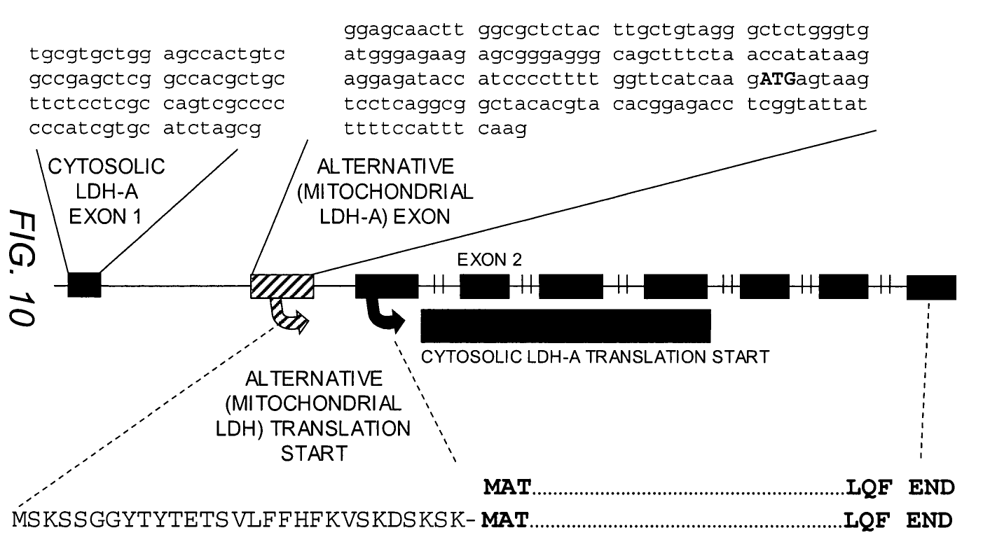

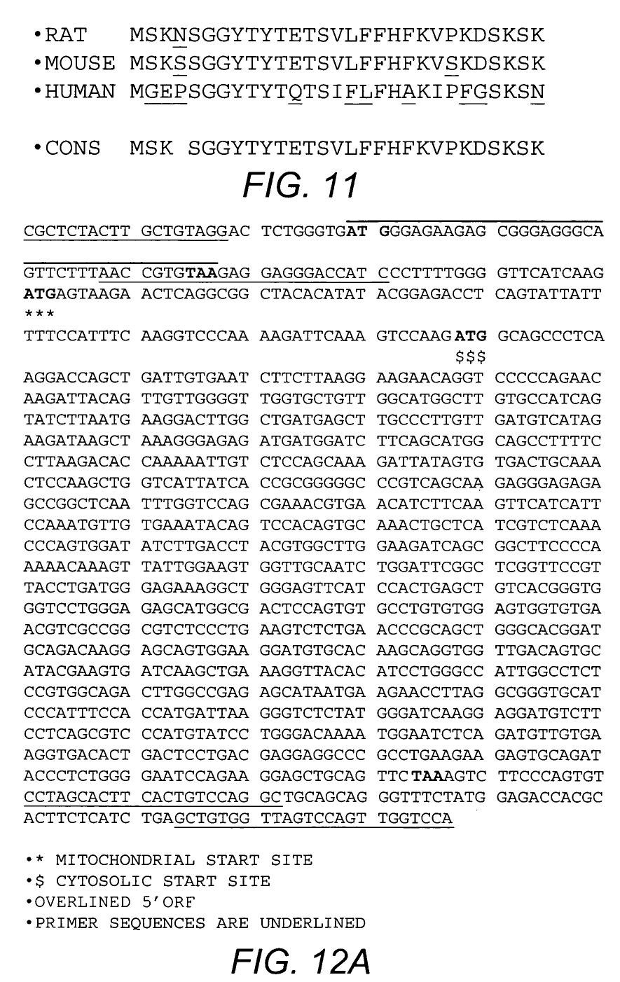

The present inventors have identified a new exon in the mouse and human LDH_{A} genes (FIG. 10) that contains an alternative translational start site and encodes an LDH_{A} isoform having a putative amino terminal mitochondrial signal peptide, which amino terminal sequences share characteristics with known mitochondrial signal peptides. The inventors have further cloned a rat liver cDNA encoding a LDH_{A} having an additional 29 amino acids at the amino terminus (SEQ ID NO:21; FIG. 10 and FIG. 11) as compared with the cytosolic form, and which shares a high degree of amino acid sequence similarity with the amino terminal sequences of the rat and human LDH.

The isolated nucleic acids of the invention can encode mitochondrial LDH_{A}, LDH_{B}, and/or LDH_{C} isoforms. The isolated nucleic acid can encode one, or more than one LDH isoform. Further, the isolated nucleic acid can encode a mitochondrial LDH from any species, as described above.

In representative embodiments of the invention, the isolated nucleic acid encoding the mitochondrial LDH will hybridize to the nucleic acid sequences encoding LDH specifically disclosed herein (i.e., SEQ ID NO:1, SEQ ID NO:3, SEQ ID NO:24 or SEQ ID NO:26) under standard conditions as known by those skilled in the art and encode a functional mitochondrial LDH as defined herein. Such sequences are intended to encompass fragments of the full-length LDH coding sequence that hybridize to the nucleic acid sequences encoding LDH specifically disclosed herein. Generally, fragments of the full-length LDH coding sequence encompassed by the present invention will encode a functional LDH polypeptide (as described below) of at least about 25, 50, 100, 150 or 200 amino acids or longer having the specified properties (e.g., as a mitochondrial targeted LDH).

To illustrate, hybridization of such sequences can be carried out under conditions of reduced stringency, medium stringency or even stringent conditions (e.g., conditions represented by a wash stringency of 35-40% Formamide with 5× Denhardt's solution, 0.5% SDS and 1×SSPE at 37° C.; conditions represented by a wash stringency of 40-45% Formamide with 5× Denhardt's solution, 0.5% SDS, and 1×SSPE at 42° C.; and/or conditions represented by a wash stringency of 50% Formamide with 5× Denhardt's solution, 0.5% SDS and 1×SSPE at 42° C., respectively) to the sequences specifically disclosed herein. See, e.g., Sambrook et al., Molecular Cloning, A Laboratory Manual (2d Ed. 1989) (Cold Spring Harbor Laboratory).

Alternatively stated, isolated nucleic acids encoding mitochondrial LDH of the invention have at least about 60%, 70%, 80%, 90%, 95%, 97%, 98% or higher sequence similarity with the isolated nucleic acid sequences specifically disclosed herein (or fragments thereof, as defined above) and encode a functional mitochondrial LDH as defined herein.

It will be appreciated by those skilled in the art that there can be variability in the nucleic acids that encode the mitochondrial LDH of the present invention due to the degeneracy of the genetic code. The degeneracy of the genetic code, which allows different nucleic acid sequences to code for the same polypeptide, is well known in the literature (see Table 1).

TABLE 1

| | |
|---|---|
| Amino Acids | Codons |

| | | | |
|---|---|---|---|
| Alanine | Ala | A | GCA GCC GCG GCT |
| Cysteine | Cys | C | TGC TGT |
| Aspartic acid | Asp | D | GAC GAT |
| Glutamic acid | Glu | E | GAA GAG |
| Phenylalanine | Phe | F | TTC TTT |
| Glycine | Gly | G | GGA GGC GGG GGT |
| Histidine | His | H | CAC CAT |
| Isoleucine | Ile | I | ATA ATC ATT |
| Lysine | Lys | K | AAA AAG |
| Leucine | Leu | L | TTA TTG CTA CTC CTG CTT |
| Methionine | Met | M | ATG |
| Asparagine | Asn | N | AAC AAT |
| Proline | Pro | P | CCA CCC CCG CCT |
| Glutamine | Gln | Q | CAA CAG |
| Arginine | Arg | R | AGA AGG CGA CGC CGG CGT |
| Serine | Ser | S | AGC ACT TCA TCC TCG TCT |
| Threonine | Thr | T | ACA ACC ACG ACT |
| Valine | Val | V | GTA GTC GTG GTT |
| Tryptophan | Trp | W | TGG |
| Tyrosine | Tyr | Y | TAC TAT |

Further variation in the nucleic acid sequence can be introduced by the presence (or absence) of non-translated sequences, such as intronic sequences and 5′ and 3′ untranslated sequences.

Moreover, the isolated nucleic acids of the invention encompass those nucleic acids encoding mitochondrial LDH polypeptides that have at least about 60%, 70%, 80%, 90%, 95%, 97%, 98% or higher amino acid sequence similarity with the polypeptide sequences specifically disclosed herein (or fragments thereof) and further encode a functional mitochondrial LDH as defined herein.

As is known in the art, a number of different programs can be used to identify whether a nucleic acid or polypeptide has sequence identity or similarity to a known sequence. Sequence identity and/or similarity can be determined using standard techniques known in the art, including, but not limited to, the local sequence identity algorithm of Smith & Waterman, Adv. Appl. Math. 2, 482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85,2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35, 351-360 (1987); the method is similar to that described by Higgins & Sharp, CABIOS 5, 151-153 (1989).

Another example of a useful algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/ README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values can be adjusted to increase sensitivity.

An additional useful algorithm is gapped BLAST as reported by Altschul et al., (1997) Nucleic Acids Res. 25, 3389-3402.

A percentage amino acid sequence identity value can be determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

The-alignment can include the introduction of gaps in the sequences to be aligned. In addition, for sequences which contain either more or fewer amino acids than the polypeptides specifically disclosed herein, it is understood that in one embodiment, the percentage of sequence identity will be determined based on the number of identical amino acids in relation to the total number of amino acids. Thus, for example, sequence identity of sequences shorter than a sequence specifically disclosed herein, will be determined using the number of amino acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

To modify the LDH amino acid sequences specifically disclosed herein or otherwise known in the art, amino acid substitutions can be based on any characteristic known in the art, including the relative similarity or differences of the amino acid side-chain substituents, for example, their hydrophobicity, hydrophilicity, charge, size, and the like. In particular embodiments, conservative substitutions (i.e., substitution with an amino acid residue having similar properties) are made in the amino acid sequence encoding LDH.

In making amino acid substitutions, the hydropathic index of amino acids can be considered. The importance of the hydropathic amino acid index in conferring interactive biologic function on a protein is generally understood in the art (see, Kyte and Doolittle, (1982) J. Mol. Biol. 157:105; incorporated herein by reference in its entirety). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, Id.), and these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is also understood in the art that the substitution of amino acids can be made on the basis of hydrophilicity. U.S. Pat. No. 4,554,101 (incorporated herein by reference in its entirety) states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (±3.0); aspartate (+3.0±1); glutamate (+3.0±1); serine (+0.3); asparagine (+0.2); glutamine (+0.2); glycine (0); threonine (−0.4); proline (−0.5±I); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

Isolated nucleic acids of this invention include RNA, DNA (including cDNAs) and chimeras thereof. The isolated nucleic acids can further comprise modified nucleotides or nucleotide analogs.

The isolated nucleic acids encoding LDH can be associated with appropriate expression control sequences, e.g., transcription/translation control signals and polyadenylation signals.

It will be appreciated that a variety of promoter/enhancer elements can be used depending on the level and tissue-specific expression desired. The promoter can be constitutive or inducible (e.g., the metalothionein promoter or a hormone inducible promoter), depending on the pattern of expression desired. The promoter can be native or foreign and can be a natural or a synthetic sequence. By foreign, it is intended that the transcriptional initiation region is not found in the wild-type host into which the transcriptional initiation region is introduced. The promoter is chosen so that it will function in the target cell(s) of interest. In embodiments of the invention, the promoter functions in pancreatic islet β-cells. The promoter can further be “specific” for these cells and tissues (i.e., only show significant activity in the specific cell or tissue type), for example, the insulin promoter for islet β-celis; the prolactin or growth hormone promoters for anterior pituitary cells.

To illustrate, a LDH coding sequence can be operatively associated with a cytomegalovirus (CMV) major immediate-early promoter, an albumin promoter, an Elongation Factor 1-α (EF1-α) promoter, a PγK promoter, a. MFG promoter, a Rous sarcoma virus promoter, an insulin promoter, or a glyceraldehyde-3-phosphate promoter.

Moreover, specific initiation signals are generally required for efficient translation of inserted protein coding sequences. These translational control sequences, which can include the ATG initiation codon and adjacent sequences, can be of a variety of origins, both natural and synthetic.

In embodiments wherein the isolated nucleic acid encoding LDH comprises an additional sequence to be transcribed, the transcriptional units can be operatively associated with separate promoters or with a single upstream promoter and one or more downstream internal ribosome entry site (IRES) sequences (e.g., the picornavirus EMC IRES sequence).

The isolated nucleic acids encoding LDH can be incorporated into a vector, e.g., for the purposes-of cloning or other laboratory manipulations, recombinant protein production, or gene delivery. Exemplary vectors include bacterial artificial chromosomes, cosmids, yeast artificial chromosomes, phage, plasmids, lipid vectors and viral vectors (described in more detail below). Nucleic acid delivery vectors are more specifically described in Section IV.

In particular embodiments, the isolated nucleic acid is incorporated into an expression vector. Expression vectors compatible with various host cells are well known in the art and contain suitable elements for transcription and translation of nucleic acids. Typically, an expression vector contains an “expression cassette,” which includes, in the 5′ to 3′ direction, a promoter, a coding sequence encoding an LDH subunit operatively associated with the promoter, and, optionally, a termination sequence including a stop signal for RNA polymerase and a polyadenylation signal for polyadenylase.

Expression vectors can be designed for expression of polypeptides in prokaryotic or eukaryotic cells. For example, polypeptides can be expressed in bacterial cells such as E. coli, insect cells (e.g., in the baculovirus expression system), yeast cells or mammalian cells. Some suitable host cells are discussed further in Goeddel, Gene Expression Technology: Methods in Enzymology 185, Academic Press, San Diego, Calif. (1990). Examples of vectors for expression in yeast S. cerevisiae include pYepSecl (Baldari et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, Calif.). Baculovirus vectors available for expression of nucleic acids to produce proteins in cultured insect cells (e.g., Sf 9 cells) include the pAc series (Smith et al., (1983) Mol. Cell. Biol. 3:2156-2165) and the pVL series (Lucklow, V.A., and Summers, M.d. (1989) Virology 170:31-39).

Examples of mammalian expression vectors include pCDM8 (Seed, (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, adenovirus 2, cytomegalovirus and Simian Virus 40.

In addition to the regulatory control sequences discussed above, the recombinant expression vector can contain additional nucleotide sequences. For example, the recombinant expression vector can encode a selectable marker gene to identify host cells that have incorporated the vector and/or may comprise another heterologous sequence of interest.

Vectors can be introduced into prokaryotic or eukaryotic cells via conventional transformation or transfection techniques. As used herein, the terms “transformation” and “transfection” refer to a variety of art-recognized techniques for introducing foreign nucleic acids (e.g., DNA) into a host cell, including calcium phosphate or calcium chloride co-precipitation, DEAE-dextran-mediated transfection, lipofection, electroporation, microinjection, DNA-loaded liposomes, lipofectamine-DNA complexes, cell sonication, gene bombardment using high velocity microprojectiles, and viral-mediated transfection. Suitable methods for transforming or transfecting host cells can be found in Sambrook et al. (Molecular Cloning: A Laboratory Manual, 2nd Edition, Cold Spring Harbor Laboratory press (1989)), and other laboratory manuals.

Often only a small fraction of cells (in particular, mammalian cells) integrate the foreign DNA into their genome. In order to identify and select these integrants, a nucleic acid that encodes a selectable marker (e.g., resistance to antibiotics) can be introduced into the host cells along with the nucleic acid of interest. Preferred selectable markers include those that confer resistance to drugs, such as G418, hygromycin and methotrexate. Nucleic acids encoding a selectable marker can be introduced into a host cell on the same vector as that comprising the nucleic acid of interest or can be introduced on a separate vector. Cells stably transfected with the introduced nucleic acid can be identified by drug selection (e.g., cells that have incorporated the selectable marker gene will survive, while the other cells die).

As discussed in more detail in the following section, the present invention further provides isolated cells comprising the isolated nucleic acids and/or polypeptides of the invention. In representative embodiments, the cell is for use in cell-based therapeutic methods, e.g., genetic manipulation of cells that are then implanted into a subject or otherwise used to deliver a therapeutic treatment to a subject.

Ill. Cell-Based Therapies.

The present invention can also be employed to enhance IS, in particular fuel-stimulated IS or glucose-stimulated IS, by a cell that is capable of secreting insulin. Such cells include insulinoma cells (e.g., INS-1 cells including their derivatives such as the highly glucose-responsive line 832/13, HIT-T15 cells, RINr1046-38 cells, MSL-G2 cells, β-celIs expressing T-antigen, typically referred to as TC cells, including bTC-3 and bTC-6 cells, MIN6 cells), primary β-cells, and “artificial β-cells” which are non-β-cells that have been engineered to produce insulin and, optionally, to respond to glucose and other fuels by secreting insulin (as described, for example, in U.S. Pat. No. 5, 744,327; incorporated herein in its entirety for its teachings of methods of creating artificial β-cells). These cells can be advantageously used in the treatment of diabetes (insulin-dependent and non-insulin-dependent), other glucose intolerant conditions, and any other condition in which it would be desirable to have enhanced IS.

In one embodiment, the invention provides an isolated cell comprising an isolated nucleic acid comprising a nucleotide sequence encoding mitochondrial LDH (as described above). In particular embodiments, the cell is capable of secreting insulin, optionally is capable of fuel-stimulated (e.g., glucose-stimulated) insulin secretion.

In other embodiments, the invention provides an isolated cell comprising an isolated nucleic acid comprising a nucleotide sequence encoding LDH, wherein fuel-stimulated (e.g., glucose-stimulated) IS by the cell is enhanced by the presence of the isolated nucleic acid as compared with the level of fuel-stimulated IS observed in a comparable cell in the absence of the isolated nucleic acid overexpressing LDH.

The cell can further comprise nucleic acids encoding a glucose transporter (e.g., GLUT 2), glucokinase and insulin. These coding sequences can be endogenous to the cell. Alternatively, or additionally, the cell can comprise an isolated nucleic acid (i.e., a foreign or recombinant nucleic acid) encoding a glucose transporter, glucokinase and/or insulin. In other words, the cell can produce the polypeptide by expression of endogenous coding sequences; alternatively or additionally, the cell can be modified by introduction of an isolated nucleic acid(s) encoding one or all of these polypeptides.

Thus, the cell can normally secrete insulin, more particularly, secrete insulin in response to glucose (e.g., a β-cell or insulinoma cell). Alternatively, the cell can be an “artificial β-cell ” that has been engineered to secrete insulin, optionally to secrete insulin in response to fuels, such as glucose.

The cell can comprise one isolated nucleic acid encoding one or more LDH subunits. Alternatively, the cell can comprise more than one isolated nucleic acid encoding one or more LDH subunits.

Sequences encoding insulin, glucose transporters and glucokinase are known in the art, see, e.g., GenBank Accession Nos. NM_{—}000207 (human insulin), NM_{—}000162 (human pancreatic glucokinase); Newgard et al., (1990) Biochem. Soc. Trans. 18:851 and Hughes et al., (1991) J. Biol. Chem. 266:4521 (islet isoform of glucokinase); NM_{—}000340 (human GLUT-2); NM_{—}003039 (human GLUT-5); NM_{—}145176 (human GLUT-12); the disclosures of which are incorporated herein in their entirety for teachings of nucleotide and amino acid sequences encoding insulin, glucose transporters or glucokinase.

This aspect of the invention is generally based on the finding that a cell that is competent to secrete insulin can be converted to a cell that exhibits fuel-stimulated IS or glucose-stimulated IS through the introduction of an isolated nucleic acid sequence encoding a functional glucose transporter protein, such as GLUT, in particular GLUT-2 (see, e.g., U.S. Pat. No. 5,744,327).

A substantial body of evidence has accumulated implicating a specific facilitated-diffusion type glucose transporter known as GLUT-2, and the glucose phosphorylating enzyme, glucokinase, in the control of glucose metabolism in islet β-cells. Both proteins are members of gene families; GLUT-2 is unique among the five-member family of glucose transporter proteins (GLUTs 1-5; Bell et al., (1990) Diabetes Care 13:198; Thorens et al., (1990) Diabetes Care 13:209) in that is has a distinctly higher Km and Vmax for glucose transport. Glucokinase (also known as hexokinase IV) is the high Km, high Vmax counterpart of GLUT-2 among the family of hexokinases (Weinhouse, (1976) Curr. Top. Cell. Regul. 11:1). Both proteins have affinities for glucose that allow dramatic changes in their activities over the physiological range of glucose. This has led to the hypothesis that these proteins work in concert as the “glucose-sensing apparatus” that modulates insulin secretion in response to changes in circulating glucose concentrations by regulating glycolytic flux (Newgard et al., (1990) Biochem. Soc. Trans. 18:851; Johnson et al., (1990) J. Biol. Chem. 265:6548).

In β-cells, glucose transport capacity is generally in excess relative to glycolytic flux. Thus, the GLUT-2 transporter likely plays a largely permissive role in the control of glucose metabolism, while glucokinase represents the true rate-limiting step (Meglasson and Matchinsky, (1986) Diabetes. Metab. Rev. 2:163; Newgard et al., (1990) Biochem. Soc. Trans. 18:851). Implicit in this formulation, however, is the prediction that severe underexpression of GLUT-2 will result in loss of glucose-stimulated insulin secretion in islets, an idea that has recently received strong experimental support from studies with spontaneous (Johnson et al., (1990) Science 250:546; Orci et al., (1990) Proc. Natl. Acad. Sci. USA 87:9953) as well as experimentally induced (Chen et al., (1990) Proc. Natl. Acad. Sci. USA 87:4088; Thorens et al., (1990) Proc. Natl. Acad. Sci. USA 87:6492) animal models of β-cell dysfunction.

Modified cells of the present invention can be derived from insulinoma cells (e.g., INS-1 cells including their derivatives such as the highly glucose-responsive line 832/13, HIT-T15 cells, RINr1046-38 cells, MSL-G2 cells, β-cells expressing T-antigen, typically referred to as TC cells, including bTC-3 and bTC-6 cells, MlN6-cells) or primary β-cells. In addition, the modified cell can be derived from a cell or cell line that is capable of forming secretory granules, in particular, an endocrine cell. Secretory granules are generally confined to mammalian cells whose main function is the synthesis and secretion of peptides, such as endocrine cells. Secretory granules are formed by budding of intracellular membranous structures known as the Golgi apparatus. Polypeptide hormones are usually synthesized as prohormones and undergo proteolytic processing to yield the shorter, mature version of the hormone.

Alternatively, in embodiments of the invention, the modified cell can be derived from a hepatoma cell line or primary hepatocytes.

Non-islet cells can be particularly advantageous in the treatment of insulin dependent diabetes mellitus, which is caused by autoimmune destruction of insulin producing β-cells. Islet transplantation has been extensively investigated as a therapeutic strategy for insulin dependent diabetes mellitus, but suffers from the difficulties associated with procuring enough tissue. This embodiment of the invention is based in part on the recognition that the problem of islet supply can be circumvented if a non-islet cell type is modified to secrete insulin in response to metabolic signals. Cultured cells are also desirable as they can be grown in unlimited quantity in vitro.

Thus, for example, the initial protein product of the insulin gene in β-cells is preproinsulin. This precursor differs from mature insulin in that it has a so-called “signal sequence” at its amino-terminus, consisting of a stretch of hydrophobic amino acids that guide the polypeptide to the rough endoplasmic reticulum. It also has a connecting peptide between the A and B chains that comprise the mature insulin molecule, this connector is known as the “C-peptide”. The preproinsulin molecule enters the lumen of the endoplasmic reticulum, in the process the hydrophobic amino-terminal “pre” region is proteolytically removed. The processed, correctly folded proinsulin molecule (still containing the C-peptide) is then transported to the Golgi apparatus. As the precursor is transported through the Golgi apparatus, enzymatic removal of the C-peptide connector begins.

Secretory granules are derived from Golgi membranes by a process of budding off and eventual separation. The resulting granule envelopes the mixture of unprocessed proinsulin and the small amount of mature insulin. Most of the processing of proinsulin to insulin occurs shortly after formation of the secretory granules by virtue of the fact that the enzymes responsible for this processing are found at highest concentration within the granules. The granules are transported to the plasma membrane surface of the cell in response to secretory stimuli such as glucose; whereupon they fuse with the plasma membrane and release their stores of the mature hormone. Some of the features of this system are (1) the secretory granules allow a supply of a particular hormone to be built up and stored for release at the time when it is needed to perform its function, and (2) the presence of processing enzymes in the granules allow efficient conversion of the precursor forms of hormones to the mature forms.

Therefore, non-β-cells used in this aspect of the invention can be derived from an endocrine secretory cell, such as a pituitary or thyroid cell. Particular endocrine cells include AtT-20 cells, which are derived from ACTH. secreting cells of the anterior pituitary gland, GH1 or the closely related GH3 cells, which are derived from growth hormone producing cells of the anterior pituitary, or other cell lines derived from this gland. AtT-20 cells are advantageous for a variety of reasons. First, these cells have been successfully modified for insulin production by stable transfection with a viral promoter/human proinsulin cDNA construct (this derivation of the AtT-20 cell line is known as AtT-20ins; both the parental AtT-20 cell line and the insulin expressing AtT-20ins cell line are available from American Type Culture Collection, 12301 Parklawn Drive, Rockville, Md. 20852). Second, AtT-20ins cells are able to process the proinsulin mRNA and preprotein to yield the correctly processed insulin polypeptide. Third, the insulin secretory response of AtT-20ins cells to analogues of cAMP compares favorably with the well-differentiated insulinoma (HIT) cell line which is derived from hamster islet β-cells. Finally, studies have shown that AtT-20ins cells contain a significant amount of the islet isoform of glucokinase, making this the only tissue other than liver or islets in which glucokinase gene expression has been reported.

AtT-20ins cells differ from islets in at least two ways. First, they do not secrete insulin in response to glucose, and second, they express the low Km GLUT-1 glucose transporter mRNA and not GLUT-2 (Hughes et al., (1991) J. Biol. Chem. 266:4521). It was proposed that the lack of glucose responsiveness in AtT-20ins cells can be explained either by deficient capacity or altered affinity of glucose uptake relative to normal islets. To test this hypothesis, AtT-20ins cells were stably transfected with GLUT-2 cDNA (e.g., to produce two cell lines designated CGT-5 and CGT-6 cells; Johnson et al., (1990) J. Biol. Chem. 265:6548). It was found that cells engineered in this way gained glucose-stimulated insulin secretion and glucose potentiation of non-glucose secretagogue stimulation, albeit with a dose-response curve that is different from normal islets.

GH1 and GH3 cells were originally derived from the same batch of cells isolated from a rat pituitary gland tumor. GH3 cells differ from GH1 cells in that they secrete more growth hormone and also secrete prolactin (both lines are available from the American Type Culture Collection). It has been shown that introduction of a recombinant preprosomatostatin gene into these cells results in secretion of the mature somatostatin peptide (Stoller et al., (1989) J. Biol. Chem. 264:6922). Processing of the endogenous preprosomatostatin gene also occurs in delta-cells of the islets of Langerhans. The finding that an islet hormone precursor can be correctly processed in growth hormone secreting cells of the anterior pituitary suggests that proinsulin processing will also occur in these cells.

A number of cell lines derived from β-cells, commonly known as insulinoma cells, can also be used in the practice of this invention and are readily available. For example, hamster insulinoma (HIT-T15) cells are well studied and are readily available from the American Type Tissue Collection. A number of rat insulinoma cell lines are also available.

The RINm5F and RINr1046-38 cell lines were derived from a radiation induced tumor of the islet β-cells (Gazdar et al., (1980) Proc. Natl. Acad. Sci. USA 77:2519; Clark et al., (1990) Endocrinology 127:277). MSL-G2 cells-were derived from a liver metastasis of an islet cell tumor. These cells require periodic passage in an animal host in order to maintain expression of their endogenous insulin gene (Madsen et al., (1988) Proc. Nat. Acad. Sci. USA 85:6652). The β-TC insulinoma cell line has been recently derived from transgenic animals injected with a T-antigen gene driven by an insulin promoter, resulting in specific expression of T-antigen in islet β-cells and consequent immortalization of these cells (Efrat et al., (1988) Proc. Natl. Acad. Sci. USA 85:9037).

RIN 1046-38 cells have been shown to express both GLUT-2 and glucokinase (Hughes et al., (1991) J. Biol. Chem. 266: 4521), and have been shown by Clark et al. (1990) Endocrinology 127:2779, to be responsive to glucose. Glucose-stimulation of insulin release from these cells is maximal at 0.5 mM glucose, a level far below the stimulatory concentration of glucose required for insulin release from normal β-cells. Recent studies suggest that this hypersensitivity to glucose in RIN 1046-38 cells may be due to high levels of hexokinase activity. Hexokinase performs the same function as glucokinase (glucose phosphorylation) but does so at much lower glucose concentrations (hexokinase has a Km for glucose of approximately 0.05 mM versus 8 mM for glucokinase). It is proposed that lowering hexokinase activity by the methods described below may render RIN cells useful for the practice of this invention.

Other illustrative insulinoma cells are INS-1 cells (including their derivatives such as the highly glucose-responsive line 832/13) and MIN6 cells. The 832/13 line is a highly differentiated model of β-cell function provided as a subclone of the rat insulinoma cell line INS-1 (Hohmeier et al., (2000) Diabetes 49:424).

As stated above, particular endocrine secretory cells for use in accordance with the present invention are AtT-20ins cells, which have been stably transfected to allow the production of correctly processed human insulin. Also as stated, a nucleotide sequence encoding the GLUT-2 isozyme can be introduced into AtT20ins cells to provide recombinant cells with a functional glucose transporter. Engineered cells that combine both of these features have been created (e.g., CTG-5 cells and CTG-6 cells), which can be used in representative embodiments of the invention.

In particular embodiments, the cells of the invention have been modified or selected for reduced hexokinase activity relative to the cell or cell line from which they were derived. There are four known isoforms of hexokinase in mammals. Hexokinases I, II and III have very low Kms (high affinities) for glucose, on the order of 0.05 mM. Hexokinase IV is glucokinase, which has a high Km for glucose of around 8-10 mM. In the islet β-cell, glucokinase is the predominant glucose phosphorylating enzyme, while in most cell lines grown in culture, the low Km hexokinase I isoform predominates. It is proposed that expression of high levels of hexokinases other than glucokinase can make the cell glucose-responsive such that insulin release is stimulated at lower concentrations of glucose than is desirable. Thus, as another aspect of the invention, the activity of hexokinase and/or glucokinase is altered to provide a relatively low hexokinase/glucokinase ratio, which can result in a more physiologic insulin response to glucose.

It has been observed that in AT-20ins cells, maximal insulin secretion occurs at a much lower glucose concentration than observed for normal islets, which do not respond at levels less than the fasting glucose concentration of approximately 4-5 mM, and which have not reached maximum secretion at the upper range of physiological glucose (10 mM). The potentiating effect of glucose on forskolin, dibutryl cAMP, or IBMX induced insulin secretion from AtT-20ins cells is also maximal at low glucose. The heightened sensitivity of GLUT-2 transfected AtT-20ins cells to both the direct and potentiating effects of glucose is reminiscent of a number of cell lines derived from insulinoma (β-cell) tumors (Praz et al., (1983) Biochem. T. 210:345; Halban et al., (1983) Biochem. J. 212:439; Giroix et al., (1985) Arch. Biochem. Biophys. 241:561; Meglasson et al., (1987) Diabetes 36:477; Clark et al., (1990) Endocrinology 127:277). For example, the rat insulinoma cell line RIN 1046-38 is responsive to glucose when studied after short periods of time in cell culture (between passages 6-17), albeit with a maximal response at sub-physiological glucose levels, as in transfected AtT-20ins cells. With longer time in culture (passage number greater than 50), all glucose-stimulated insulin secretion is lost (Clark et al., (1990) Endocrinology 127:277). Low passage RIN 1046-38 cells contain both glucokinase and GLUT-2, but lose expression of these genes when studied at higher passages.

A more physiologic glucose response can be achieved by reducing hexokinase activity in engineered cells of the present invention. Reductions in hexokinase can be determined by measuring enzymatic activity or by measuring proteins levels, both of which can be carried out by techniques well-known in the art. In particular embodiments, the level of cellular hexokinase activity and/or hexokinase protein is reduced by at least about 40%, 50%, 60%, 70%, 80%, 90%, 95% or more.

Various approaches can be taken to reduce cellular hexokinase activity or protein. One approach involves the introduction of an antisense nucleotide sequence. The term “antisense nucleotide sequence,” as used herein, refers to a nucleotide sequence that is complementary to a specified DNA or RNA sequence. Antisense RNA sequences and nucleic acids that express the same can be made in accordance with conventional techniques. See, e.g., U.S. Pat. No. 5,023,243 to Tullis; U.S. Pat. No. 5,149,797 to Pederson et al.

As one example, an antisense RNA can be expressed by juxtaposition of a coding sequence for hexokinase (or a portion thereof) in a reverse orientation behind a suitable promoter, such that an antisense RNA molecule is produced. This antisense construct is then introduced into the cell and, upon its expression, the production of hexokinase is reduced in the cell. Alternatively, an antisense nucleotide sequence can be directly introduced into the cell by other techniques, such as electroporation. After appropriate selection to obtain cells that have stably incorporated the antisense nucleotide sequence (e.g., by stable incorporation into their genome or by stable maintenance of episomal constructs), expression of the antisense mRNA can be evaluated (for example, by hybridization to labeled sense RNA, e.g., prepared with the pGEM vector system; Promega). One can assess whether the presence of the antisense nucleotide sequence affects the production of hexokinase polypeptide or hexokinase enzymatic activity using known techniques in the art.

Those skilled in the art will appreciate that it is not necessary that the antisense nucleotide sequence be fully complementary to the target sequence as long as the degree of sequence similarity is sufficient for the antisense nucleotide sequence to hybridize to its target and reduce production of hexokinase. As is known in the art, a higher degree of sequence similarity is generally required for short antisense nucleotide sequences, whereas a greater degree of mismatched bases will be tolerated by longer antisense nucleotide sequences. Alternatively stated, antisense nucleotide sequences of the invention have at least about 60%, 70%, 80%, 90%, 95%, 97%, 98% or higher sequence similarity with the hexokinase coding sequence (or portions thereof) and reduce the level of hexokinase polypeptide production (as defined above).

The length of the antisense nucleotide sequence (i.e., the number of nucleotides therein) is not critical as long as it binds selectively to the intended location and reduces transcription and/or translation of the target sequence (e.g., by at least about 40%, 50%, 60%, 70%, 80%, 90%, 95% or more), and can be determined in accordance with routine procedures. In general, the antisense nucleotide sequence will be from about eight, ten or twelve nucleotides in length up to about 20, 30, 50, 60 or 70 nucleotides, or longer, in length.

An antisense nucleotide sequence can be constructed using chemical synthesis and enzymatic ligation reactions by procedures known in the art. For example, an antisense nucleotide sequence can be chemically synthesized using naturally occurring nucleotides or various modified nucleotides designed to increase the biological stability of the molecules or to increase the physical stability of the duplex formed between the antisense and sense nucleotide sequences, e.g., phosphorothioate derivatives and acridine substituted nucleotides can be used. Examples of modified nucleotides which can be used to generate the antisense nucleotide sequence include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiourid ine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleotide sequence can be produced using an expression vector into which a nucleic acid has been cloned in an antisense orientation (ie., RNA transcribed from the inserted nucleic acid will be of an antisense orientation to a-target nucleic acid of interest).

The antisense nucleotide sequences of the invention further include nucleotide sequences wherein at least one, or all, or the internucleotide bridging phosphate residues are modified phosphates, such as methyl phosphonates, methyl phosphonothioates, phosphoromorpholidates, phosphoropiperazidates and phosphoramidates. For example, every other one of the internucleotide bridging phosphate residues can be modified as described. In another non-limiting example, the antisense nucleotide sequence is a nucleotide sequence in which one, or all, of the nucleotides contain a 2′ loweralkyl moiety (e.g., C₁-C₄, linear or branched, saturated or unsaturated alkyl, such as methyl, ethyl, ethenyl, propyl, 1-propenyl, 2-propenyl, and isopropyl). For example, every other one of the nucleotides can be modified as described. See also, Furdon et al., (1989) Nucleic Acids Res. 17, 9193-9204; Agrawal et al., (1990) Proc. Natl. Acad. Sci. USA 87, 1401-1405; Baker et al., (1990) Nucleic Acids Res. 18, 3537-3543; Sproat et al., (1989) Nucleic Acids Res. 17, 3373-3386; Walder and Walder, (1988) Proc. Natl. Acad. Sci. USA 85, 5011-5015; incorporated by reference herein in their entireties for their teaching of methods of making antisense molecules, including those containing modified nucleotide bases).

RNA interference (RNAi) provides another approach for reducing hexokinase activity. The RNAi can be directed against the hexokinase coding sequence in the cell or any other sequence that results in a reduction in hexokinase activity.

RNAi is a mechanism of post-transcriptional gene silencing in which double-stranded RNA (dsRNA) corresponding to a coding sequence of interest is introduced into a cell or an organism, resulting in degradation of the corresponding mRNA. The RNAi effect persists for multiple cell divisions before gene expression is regained. RNAi is therefore a powerful method for making targeted knockouts or “knockdowns” at the RNA level. RNAi has proven successful in human cells, including human embryonic kidney and HeLa cells (see, e.g., Elbashir et al., Nature (2001) 411:494-8). In one embodiment, silencing can be induced in mammalian cells by enforcing endogenous expression of RNA hairpins (see Paddison et al., (2002), PNAS USA 99:1443-1448). In another embodiment, transfection of small (e.g., 21-23 nt) dsRNA specifically inhibits nucleic acid expression (reviewed in Caplen, (2002) Trends in Biotechnology 20:49-51).

The mechanism by which RNAi achieves gene silencing has been reviewed in Sharp et al, (2001) Genes Dev 15: 485-490; and Hammond et al., (2001) Nature Rev Gen 2:110-119).

RNAi technology utilizes standard molecular biology methods. To illustrate, dsRNA corresponding to all or a part of a target coding sequence to be inactivated can be produced by standard methods, e.g., by simultaneous transcription of both strands of a template DNA (corresponding to the target sequence) with T7 RNA polymerase. Kits for production of dsRNA for use in RNAi are available commercially, e.g., from New England Biolabs, Inc. Methods of transfection of dsRNA or plasmids engineered to make dsRNA are routine in the art.

Silencing effects similar to those produced by RNAi have been reported in mammalian cells with transfection of a mRNA-cDNA hybrid construct (Lin et al., (2001) Biochem Biophys Res Commun 281:639-44), providing yet another strategy for silencing a coding sequence of interest.

An alternative approach to the reduction of hexokinase action is through homologous recombination. Homologous recombination relies, like antisense, on the tendency of nucleic acids to base pair with complementary sequences. In this instance, the base pairing serves to facilitate the interaction of two separate,nucleic acid molecules so that strand breakage and repair can take place. In other words, the “homologous” aspect of the method relies on sequence homology to bring two complementary sequences into close proximity, while the “recombination” aspect provides for one complementary sequence to replace the other by virtue of the breaking of certain bonds and the formation of others.

Put into practice, homologous recombination can be used as follows. First, a target coding sequence is selected within the host cell. Sequences homologous to the target nucleic acid are then included in a genetic construct, along with a mutation that will render the target coding sequence inactive (e.g., a stop codon, interruption, etc.). The homologous sequences flanking the inactivating mutation are said to “flank” the mutation. Flanking, in this context, means that target homologous sequences are located both upstream. (5′) and downstream (3′) of the mutation. These sequences will generally correspond to some sequences upstream and downstream of the target coding sequence. The construct is then introduced into the cell, thus permitting recombination between the cellular sequences and the construct.

It is common to include within the genetic construct a nucleotide sequence encoding a positive selectable marker to facilitate selection for recombinants. The positive selectable marker permits selection of cells that have integrated the construct into their genomic DNA by conferring resistance to the selection agent, e.g., a biostatic or biocidal drug. In addition, a heterologous nucleotide sequence (e.g., encoding a polypeptide of interest) can advantageously be included within the genetic construct and thereby be stably introduced into the cell.

Thus, using this kind of construct, it is possible, in a single recombination event, to (i) “knock out” an endogenous coding sequence, (ii) provide a selectable marker for identifying such an event, and (iii) introduce a heterologous nucleotide sequence for expression.

Another refinement of the homologous recombination approach involves the use of a “negative” selectable marker. This marker, unlike the positive selectable marker discussed above, causes death of cells that express the marker. Thus, it is used to identify undesirable recombination events. When seeking to select homologous recombinants using a selectable marker, it is sometimes difficult in the initial screening step to identify proper homologous recombinants from recombinants generated from random, non-sequence specific events. By including a negative selectable marker in the construct, but located outside of the flanking regions, one can select against many random recombination events that result in incorporation of the negative selectable marker. Homologous re combination should not introduce the negative selectable marker into the genome, as it is outside of the flanking sequences. In particular embodiments, the genetic construct also contains a nucleotide sequence encoding a positive selectable marker as described above.

In one particular embodiment, the genetic construct comprises a nucleotide sequence encoding the GLUT-2 or GLUT-5 glucose transporter as a negative selectable marker and also comprises a positive selectable marker. Application of a positive selection agent to such cells will permit isolation of recombinants, but further application of streptozotocin (glucopyranose, 2-deoxy-2-[3-methyl-e-nitrosourido-D]; STZ) to the cells will result in killing of non-homologous recombinants because the incorporated nucleotide sequence encoding the GLUT-2 or GLUT-5 transporter will render the cells susceptible to STZ treatment. This method presupposes that the host cell does not produce endogenous GLUT-2 or GLUT-5.

On the other hand, site-specific recombination, relying on the homology between the genetic construct and the target coding sequences, will result in incorporation of the positive selectable marker only; the GLUT-2 or GLUT-5 coding sequences will not be introduced by the homologous recombination event because they are positioned outside of the flanking sequences. These cells will be resistant to the positive selection agent and are insensitive to STZ. This double-selection procedure yields recombinants that lack the target coding sequence and express the positive selection marker.

Ribozymes provide still another approach for reducing cellular hexokinase activity. Ribozymes are RNA-protein complexes that cleave nucleic acids in a site-specific fashion. Ribozymes have specific catalytic domains that possess endonuclease activity (Kim et al., (1987) Proc. Natl. Acad. Sci. USA 84:8788; Gerlach et al., (1987) Nature 328:802; Forster and Symons, (1987) Cell 49:211). For example, a large number of ribozymes accelerate phosphoester transfer reactions with a high degree of specificity, often cleaving only one of several phosphoesters in an oligonucleotide substrate (Michel and Westhof, (1990) J. Mol. Biol. 216:585; Reinhold-Hurek and Shub, (1992) Nature 357:173). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, (1989) Nature 338:217). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression can be particularly suited to therapeutic applications (Scanlon et al., (1991)Proc. Natl. Acad. Sci. USA 88:10591; Sarver et al., (1990) Science 247:1222; Sioud et al., (1992) J. Mol. Biol. 223:831).

Genomic site-directed mutagenesis with oligonucleotides is yet another approach for reducing hexokinase activity in the cell. Through analysis of radiation-sensitive mutants of Ustilago maydis, several genes have been characterized that participate in DNA repair (Tsukuda et al., (1989) Gene 85:335; Bauchwitz and Holloman, (1990) Gene 96:285). One such gene, REC2, encodes a protein that catalyzes homologous pairing between complementary nucleic acids and is required for a functional recombinational repair pathway (Kmiec et al., (1994) Mol. Cell. Biol. 14:7163; Rubin et al., (1994) Mol. Cell. Biol. 14:6287). In vitro characterization of the REC2 protein showed that homologous pairing was more efficient between RNA-DNA hybrids than the corresponding DNA duplexes (Kmiec et al, (1994) Mol. Cell. Biol. 14:7163;; PCT, WO 96/22364). However, efficiency in pairing between DNA:DNA duplexes could be enhanced by increasing the length of the DNA oligonucleotides (Kmiec et al., (1994) Mol. Cell. Biol. 14:7163). These observations led investigators to test the use of chimeric RNA-DNA oligonucleotides (RDOs) in the targeted modification of genes in mammalian cell lines (Yoon et al., (1996) Proc. Natl. Acad. Sci. USA 93:2071; Cole-Strauss et al., (1996) Science 273:1386; PCT W095/15972). The RNA-DNA oligonucleotides contained self-annealing sequences such that double-hairpin capped ends are formed. This feature is believed to increase the in vivo half-life of the RDO by decreasing degradation by helicases and exonucleases. Further, the RDOs contained a single base pair that differs from the target sequence and otherwise aligns in perfect register. It is believed that the single mismatch is recognized by the DNA repair enzymes. The RDOs further contained RNA residues modified by 2′-O-methylation of the ribose sugar, making the RDO resistant to degradation by ribonuclease activity (Monia et al., (1993) J. Biol. Chem. 268:14541).

Two separate experimental systems have been used to test the use of RDOs for targeted disruption in mammalian cell lines. In one system, RDOs were used to target and correct an alkaline phosphatase cDNA that was maintained as an episomal DNA construct in Chinese hamster ovary cells. An inactive form of alkaline phosphatase was converted to a wild-type form with an efficiency of about 30% (Yoon et al., (1996) Proc. Natl. Acad. Sci. USA 93:2071). In a second system, a genetic mutation within the chromosomal DNA was targeted and corrected. A lymphoid blast cell line was derived from a patient with sickle cell disease who was homozygous for a point mutation in the beta-globin gene. The overall frequency of gene conversion from the mutant to the wild-type form was relatively high and was found to be dose-dependent on the concentration of the RDOs (Cole-Strauss et al., (1996) Science 273:1386).

As yet another approach, random integration can be used to reduce production of hexokinase in the host cell. Although lacking the specificity of homologous recombination, there are situations in which random integration can be used as a method of knocking out a particular endogenous coding sequence. Unlike homologous recombination, the recombination is completely random, i.e., does not depend on or is not effected by base-pairing of complementary nucleic acid sequences. Random integration is like homologous recombination, however, in that a genetic construct, optionally containing a heterologous nucleotide sequence to be introduced into the cell and a selectable marker, intergrates into the target cell genomic DNA via strand breakage and reformation.

Because of the lack of sequence specificity, the chances of any given recombinant integrating into the target coding sequence are greatly reduced. In addition, second site integrants can result in loss of expression of the heterologous coding sequence that is introduced into the cell. For example, the second locus can encode a transcription factor needed for expression of the heterologous sequence of interest, etc. As a result, it may be necessary to screen many (e.g., hundreds of thousands) of drug-resistant recombinants before a desired mutant is found. Screening can be facilitated by examining recombinants for expression of the target coding sequence using immunologic or functional tests.

In addition to, or as an alternate to, decreasing hexokinase activity, the glucokinase activity of the cell can be increased by introducing an isolated nucleic acid encoding glucokinase into the cell, for example using a cDNA clone for the islet isoform of glucokinase (Newgard et al., (1990) Biochem. Soc. Trans. 18:851; Hughes et al., (1991) J. Biol. Chem. 266:4521). For example, although glucokinase activity is present in AtT-20ins cells, the activity of 0.7 U/g protein is only about 25% of the activity in normal islet cells, which contain approximately 3.1 U/g protein. Thus, it can be advantageous to increase production of glucokinase in the host cell. In other particular embodiments, the cell does not contain an isolated nucleic acid that encodes glucokinase and/or otherwise overexpress glucokinase.

In other embodiments, the host cell is a secretory cell (including endocrine cells) in which production of the endogenous polypeptide that is normally produced/secreted by the host cell has been reduced or blocked (see, e.g., U.S. Pat. No. 6,194,176; incorporated herein by reference in its entirety for teachings of methods of expressing a secreted polypeptide in a secretory cell in which production of the endogenous secreted polypeptide is reduced or blocked). By reducing or blocking production of the endogenous secreted polypeptide, the capacity of the secretory cell to produce and secrete the polypeptide of interest (i.e., insulin) can be increased. According to this embodiment, the cell can be a non-β-cell (i.e., is a cell that does not normally secrete insulin) that has been genetically modified to produce insulin from an isolated nucleic acid that has been introduced into the cell by recombinant nucleic acid techniques. Production of the endogenous secreted polypeptide can be reduced by any level that is desirable in the practice of the present invention, e.g., by at least about 25%, 40%, 50%, 60%, 70%, 80%, 90%, 95%, 98% or more. In particular embodiments, the production of the endogenous secreted polypeptide is blocked such that no detectable endogenous polypeptide is produced by the cell. Methods of detecting polypeptides are well-known in the art (e.g., by Western blot analysis).

In particular embodiments, the host cell is a β-cell or cell line (e.g., an insulinoma cell line) that produces insulin by expression from an endogenous coding sequence. According to this embodiment, production of insulin from the endogenous coding sequence is reduced or blocked, and a foreign isolated nucleic acid encoding insulin is introduced into the cell.

Inhibiting or blocking the production of an endogenous secreted. polypeptide can be carried out by any method known in the art, including the methods discussed above with respect to methods of inhibiting hexokinase activity in the cell. Such techniques include but are not limited to antisense nucleic acids, RNAi, ribozymes, genomic site-directed mutagenesis, homologous recombination, and random integration.

The capacity of the cell to produce insulin can also be enhanced by overexpressing proteins involved in protein processing in the cell, such as the endoproteases PC2 and PC3 (Steiner et al., (1992) J. Biol. Chem. 267:23435). In addition, in particular embodiments, the host cell can be advantageously modified to express an isolated nucleic acid encoding the islet β-cell specific transcription factor Insulin Promoter Factor 1 (IPF-1; Ohisson et al., (1993) EMBO J. 12:425 1). IPF-1 is a homeodomain-containing transcription factor proposed to play an important role in both pancreatic development and insulin gene expression in mature islet β-cells (Ohlsson et al., (1993) EMBO J. 12:4251, Leonard et al., (1993) Mol. Endocrinol. 7:1275, Miller et al., (1994) EMBO J. 13:1145, Kruse et al., (1993) Genes and Dev. 7:774). In embryos, IPF-1. is expressed prior to expression of nucleotide sequence encoding hormones by the islet cells and is restricted to positions within the primitive foregut where the pancreas will later form. Indeed, mice in which the IPF-1 gene is disrupted by targeted knockout do not form a pancreas (Jonsson etal., (1994) Nature,371:606). Later in pancreatic development, as the different cell types of the pancreas start to emerge, IPF-1 expression becomes restricted predominantly to β-cells. IPF-1 binds to TAAT consensus motifs contained within the FLAT E and P1 elements of the insulin enhancer/promoter, whereupon it interacts with other transcription factors to activate insulin gene transcription (Peers et al., (1994) Mol. Endocrinol. 8:1798).

Stable introduction of an isolated nucleic acid encoding IPF-1 into the modified cells of the invention can increase expression of nucleotide sequences operably associated with the insulin enhancer/promoter. In addition, because IPF-1 appears to be involved in β-cell maturation, stable expression (or overexpression) of IPF-1 in β-cell lines can cause these relatively dedifferentiated β-cells to regain a more differentiated phenotype and function.

As another embodiment, the present invention is directed to a method of providing fuel-responsive insulin secreting capability (e.g., glucose-responsive insulin secreting capability) to an animal (e.g., a mammal) in need thereof. In particular embodiments, the methods of the invention comprise implanting modified cells (as described above), which secrete insulin in response to an elevation in fuel concentrations (e.g., glucose concentrations) into such an animal. Techniques presently in use in the art for the implantation of islets will be applicable to implantation of cells engineered in accordance with the present invention.

One representative method involves the encapsulation of cells in a biocompatible coating. In this approach, cells are entrapped in a capsular coating that protects the encapsulated cells from immunological responses, and also serves to prevent uncontrolled proliferation and spread of the cells. An exemplary encapsulation technique involves encapsulation with alginate-polylysine-alginate. In particular embodiments, capsules made employing this technique generally contain several hundred cells and have a diameter of approximately 1 mm.

Engineered cells can be implanted using the alginate-polylysine encapsulation technique of O'Shea and Sun (1986), Diabetes 35:943, with modifications as recently described by Fritschy et al. (1991) Diabetes 40:37. According to this method, the engineered cells are suspended in 1.3% sodium alginate and encapsulated by extrusion of drops of the cell/alginate suspension through a syringe into CaCl₂. After several washing steps, the droplets are suspended in polylysine and rewashed. The alginate within the capsules is then reliquified by suspension in 1 ml EGTA and then rewashed with Krebs balanced salt buffer. Each capsule should contain several hundred cells and have a diameter of approximately one mm.

Implantation of encapsulated islets into animal models of diabetes by the above method has been shown to significantly increase the period of normal glycemic control, by prolonging xenograft survival compared to unencapsulated islets (O'Shea and Sun (1986), Diabetes 35:943; Fritschy, et al. (1991) Diabetes 40:37). Also, encapsulation can prevent uncontrolled proliferation of clonal cells. Capsules containing cells are implanted (e.g., from about 500, 1,000 or 2,000 cells to about 5,000, 10,000 or 20,000 cells/animal) intraperitoneally and blood samples taken daily for monitoring of blood glucose and insulin.

An alternative approach is to seed Amicon fibers with engineered cells. The cells become enmeshed in the fibers, which are semipermeable, and are thus protected in a manner similar to the micro encapsulates (Altman et al., (1986) Diabetes 35:625).

After successful encapsulation or fiber seeding, the cells, generally approximately 1,000-10,000, can be implanted intraperitoneally, usually by injection into the peritoneal cavity through a large gauge needle (23 gauge).

A variety of other encapsulation technologies have been developed that are applicable to the practice of the present invention (see, e.g., Lacy et al., (1991), Science, 254:1782-1784; Sullivan et al., Science, 252:718-721; PCT publications WO 91/10470; WO 91/10425; WO 90/15637; WO 90/02580; WO 8901967; U.S. Pat. No. 5,011,472; U.S. Pat. No. 4,892,538; each of the foregoing being incorporated by reference herein in its entirety for teachings of encapsulation technologies). The company Cytotherapeutics has developed encapsulation technologies that are now commercially available and are of use in the application of the present invention. A vascular device has also been developed by Biohybrid, of Shrewsbury, Mass., which has application to the technology of the present invention.

With respect to implantation methods which can be employed to provide a fuel-responsive (e.g., glucose-responsive) insulin secreting capability to a mammal, particular advantages can be found in the methods recently described by Lacy et al. (1991), Science, 254:1782-1784, and Sullivan et al., (1991) Science, 252:718-721, each incorporated herein by reference in its entirety for teachings of implantation methods. These concern, firstly, the subcutaneous xenograft of encapsulated islets, and secondly, the long-term implantation of islet tissue in an “artificial pancreas” which can be connected to the vascular system as an arteriovenous shunt. These implantation methods can be advantageously adapted for use with the present invention by employing modified cells, as disclosed herein, in the place of the “islet tissue” described in these publications.

Lacy and colleagues, (1991), Science, 254:1782-1784, encapsulated rat islets in hollow acrylic fibers and immobilized these in alginate hydrogel. Following intraperitoneal transplantation of the encapsulated islets into diabetic mice, normoglycemia was reportedly restored. Similar results were also obtained using subcutaneous implants that had an appropriately constructed outer surface on the fibers. The modified cells of the present invention can also be straightforwardly “transplanted” into a mammal by similar subcutaneous injection.

The development of a biohybrid perfused “artifical pancreas”, which encapsulates islet tissue in a selectively permeable membrane, has also been reported (Sullivan et al., (1991) Science, 252:718-721). In these studies, a tubular semi-permeable membrane was coiled inside a protecting housing to provide a compartment for the islet cells. Each end of the membrane was then connected to an arterial polytetrafluoroethylene (PTFE) graft that extended beyond the housing and joined the device to the vascular system as an arteriovenous shunt. The implantation of such a device containing islet allografts into pancreatectomized dogs was reported to result in the control of fasting glucose levels. Grafts of this type encapsulating modified cells described herein can also be used in accordance with the present invention.

An alternate approach to encapsulation is to simply inject the modified cells into the scapular region or peritoneal cavity of diabetic mice or rats, where these cells are reported to form tumors (Sato et al., (1962) Proc. Natl. Acad. Sci. USA 48:1184). This approach is beneficial for testing the function of cells in experimental animals but is not generally not applicable as a strategy for treating human diabetes.

Cells that have been taken from the subject, and modified ex vivo as described herein, can also be used. Dr. Richard Mulligan and his colleagues at the Massachusetts Institute of Technology have used retrovirus vectors for the purposes of introducing foreign nucleotide sequences into bone marrow cells (see, e.g, Cone et al., (1984) Proc. Natl. Acad. Sci. USA 81: 6349; Danos et al., (1988) Proc. Natl. Acad. Sci. USA 85:6460). The cells of the bone marrow are derived from a common progenitor, known as pluripotent stem cells, which give rise to a variety of blood borne cells including erythrocytes, platelets, lymphocytes, macrophages, and granulocytes. Interestingly, some of these cells, particularly the macrophages, are capable of secreting peptides such as tumor necrosis factor and interleukin 1 in response to specific stimuli. There is also evidence that these cells contain granules similar in structure to the secretory granules of β-cells (Stossel, (1987) in The Molecular Basis of Blood Diseases, Chapter 14, pp. 499-533, W.B. Saunders Co. Philadelphia, Pa. In particular embodiments, this approach circumvents the need for encapsulation of cells, since the patient's own bone marrow cells would be used for ex vivo manipulation and then re-implanted. These cells can differentiate (i.e., into a macrophage) and circulate in the blood where they will respond to elevations in circulating glucose by secreting insulin.

Other vector systems for genetically-modifying the secretory cells of the invention are described in more detail in Section IV below. Methods of removing cells from subjects for delivery of nucleic acids ex vivo, followed by introduction back into the subject are known in the art (see, e.g., U.S. Pat. No. 5,399,346 for the teaching of ex vivo viral vector administration).

The present invention finds use in veterinary and medical applications. Suitable subjects include both avians and mammals, with mammals being preferred. The term “avian” as used herein includes, but is not limited to, chickens, ducks, geese, quail, turkeys and pheasants. The term “mammal” as used herein includes, but is not limited to, humans, simians and other non-human primates, bovines, ovines, caprines, equines, felines, canines, lagomorphs, rats, mice, etc. In particular embodiments, the subject is a diabetic subject (non-insulin dependent or insulin dependent diabetes mellitus), an obese subject, or a subject with impaired glucose intolerance. Human subjects include neonates, infants, juveniles, and adults. In other representative embodiments, the subject is an animal model of diabetes, obesity or impaired glucose tolerance. In other particular embodiments, the subject is a subject in need of the therapeutic methods of the invention, e.g., because the subject is diagnosed with a glucose intolerant conditions such as diabetes, is suspected of having such a condition, or is at risk of developing such a condition.

As another aspect, the present invention provides a pharmaceutical composition comprising a modified cell or cells of the invention in a pharmaceutically acceptable carrier.

In still other embodiments, the present invention provides a pharmaceutical composition comprising a vector of the invention in a pharmaceutically acceptable carrier.

By “pharmaceutically acceptable” it is meant a material that is not biologically or otherwise undesirable, i.e., the material can be administered to a subject without causing any undesirable biological effects such as toxicity.

The formulations of the invention can optionally comprise medicinal agents, pharmaceutical agents, carriers, adjuvants, dispersing agents, diluents, and the like. See, e.g., Remington, The Science And Practice of Pharmacy (9^{th} Ed. 1995).

IV. Deliverv Vectors.

The cells of the invention have been modified (i.e., genetically engineered) by introduction of an isolated nucleic acid encoding LDH. In embodiments of the invention, the nucleic acid can be expressed transiently in the target cell; typically, however, the nucleic acid is stably incorporated into the target cell, for example, by integration into the genome of the cell or by persistent expression from stably maintained episomes (e.g., derived from Epstein Barr Virus).

The invention can be carried out by introducing a nucleic acid encoding a single LDH isoform (e.g., the A isoform) into a cell or subject, or alternatively, by co-introduction of nucleic acids encoding the A; B and/or C isoforms. Co-introduction can involve introduction of a single vector encoding LDH A, B and/or C isoforms or separate vectors encoding each of the subunits.

As discussed above, the invention encompasses methods of providing fuel-stimulated IS (e.g., glucose-stimulated IS) capability to an animal subject (e.g., an avian or mammalian subject), comprising implanting into an animal subject in need of insulin secreting capability a therapeutically effective amount of a population of cells according to the present invention, optionally positioning the cells in a semi-permeable membrane.

Berman & Newgard, (1998) Biochemistry 37:4543, found that overexpression of glucokinase has a limited metabolic impact in islet cells as compared with hepatocytes and suggested that this disparity was related to limited flux through the distal steps of glycolysis in the islet cell (see, e.g., paragraph spanning pages 4551-4552). This hypothesis suggested further research to investigate whether “simultaneous overexpression” of LDH would “be an approach to unveiling the full metabolic impact of glucokinase overexpression, and whether such a maneuver will enhance glucose-stimulated insulin secretion” (p. 4552). This publication does not appreciate that LDH overexpression alone is potentiating for fuel-stimulated IS. Further, the importance of a compartmentalized pool (e.g., mitochondrial) of LDH in enhancing fuel-stimulated IS was not recognized. In representative embodiments of the present invention, an isolated nucleic acid encoding LDH is introduced into a cell, wherein the cell does not comprise an isolated nucleic acid encoding glucokinase and/or does not overexpress glucokinase.

It will be apparent to those skilled in the art that any suitable vector can be used to deliver the isolated nucleic acids of this invention to the target cell(s) of interest. The choice of delivery vector can be made based on a number of factors known in the art, including age and species of the target host, level and persistence of expression desired, the target cell, route of delivery, size of the isolated nucleic acid, safety concerns, and the like.

Suitable vectors include virus vectors (e.g., retrovirus, alphavirus; vaccinia virus; adenovirus, adeno-associated virus, or herpes simplex virus), lipid vectors, poly-lysine vectors, synthetic polyamino polymer vectors that are used with nucleic acid molecules, such as plasmids, and the like.

Any viral vector that is known in the art can be used in the present invention. Examples of such viral vectors include, but are not limited to vectors derived from: Adenoviridae; Birnaviridae; Bunyaviridae; Caliciviridae, Capillovirus group; Carlavirus group; Carmovirus virus group; Group Caulimovirus; Closterovirus Group; Commelina yellow mottle virus group; Comovirus virus group; Coronaviridae; PM2 phage group; Corcicoviridae; Group Cryptic virus; group Cryptovirus; Cucumovirus virus group Family ([PHgr]6 phage group; Cysioviridae; Group Carnation ringspot; Dianthovirus virus group; Group Broad bean wilt; Fabavirus virus group; Filoviridae; Flaviviridae; Furovirus group; Group Germinivirus; Group Giardiavirus, Hepadnaviridae; Herpesviridae; Hordeivirus virus group; Illarvirus virus group; Inoviridae; Iridoviridae; Leviviridae; Lipothrixviridae; Luteovirus group; Marafivirus virus group; Maize chlorotic dwarf virus group; icroviridae; Myoviridae; Necrovirus group; Nepovirus virus group; Nodaviridae; Orthomyxoviridae; Papovaviridae; Paramyxoviridae; Parsnip yellow fleck virus group; Partitiviridae; Parvoviridae; Pea enation mosaic virus group; Phycodnaviridae; Picomaviridae; Plasmaviridae; Prodoviridae; Polydnaviridae; Potexvirus group; Potyvirus; Poxviridae; Reoviridae; Retroviridae; Rhabdoviridae; Group Rhizidiovirus; Siphoviridae; Sobemovirus group; SSV 1-Type Phages; Tectiviridae; Tenuivirus; Tetraviridae; Group Tobamovirus; Group Tobravirus; Togaviridae; Group Tombusvirus; Group Torovirus; Totiviridae; Group Tymovirus; and plant virus satellites.

Protocols for producing recombinant viral vectors and for using viral vectors for nucleic acid delivery can be found in Current Protocols in Molecular Biology, Ausubel, F. M. et al. (eds.) Greene Publishing Associates, (1989) and other standard laboratory manuals (e.g., Vectors for Gene Therapy. In: Current Protocols in Human Genetics. John Wiley and Sons, Inc.: 1997).

Particular examples of viral vectors are those previously employed for the delivery of nucleic acids including, for example, retrovirus, adenovirus, AAV, herpes virus, and poxvirus vectors.

In certain embodiments of the present invention, the delivery vector is an adenovirus vector. The term “adenovirus” as used herein is intended to encompass all adenoviruses, including the Mastadenovirus and Aviadenovirus genera. To date, at least forty-seven human serotypes of adenoviruses have been identified (see, e.g., FIELDS et al., VIROLOGY, volume 2, chapter 67 (3d ed., Lippincott-Raven Publishers). Preferably, the adenovirus is a serogroup C adenovirus, still more preferably the adenovirus is serotype 2 (Ad2) or serotype 5 (Ad5).

The various regions of the adenovirus genome have been mapped and are understood by those skilled in the art (see, e.g., FIELDS et al., VIROLOGY, volume 2, chapters 67 and 68 (3d ed., Lippincoft-Raven Publishers). The genomic sequences of the various Ad serotypes, as well as the nucleotide sequence of the particular coding regions of the Ad genome, are known in the art and can be accessed, e.g., from GenBank and NCBI (see, e.g., GenBank Accession Nos. J0917, M73260, X73487, AF108105, L19443, NC 003266 and NCBI Accession Nos. NC 001405, NC 001460, NC 002067, NC 00454).

Those skilled in the art will appreciate that the inventive adenovirus vectors can be modified or “targeted” as described in Douglas et al., (1996) Nature Biotechnology 14:1574; U.S. Pat. No. 5,922,315 to Roy et al.; U.S. Pat. No. 5,770,442 to Wickham et al.; and/or U.S. Pat. No. 5,712,136 to Wickham et al.

An adenovirus vector genome or rAd vector genome will typically comprise the Ad terminal repeat sequences and packaging signal. An “adenovirus particle” or “recombinant adenovirus particle” comprises an adenovirus vector genome or recombinant adenovirus vector genome, respectively, packaged within an adenovirus capsid. Generally, the adenovirus vector genome is most stable at sizes of about 28 kb to 38 kb (approximately 75% to 105% of the native genome size). In the case of an adenovirus vector containing large deletions and a relatively small heterologous nucleic acid of interest, “stuffer DNA” can be used to maintain the total size of the vector within the desired range by methods known in the art.

Normally, adenoviruses bind to a cell surface receptor (CAR) of susceptible cells via the knob domain of the fiber protein on the virus surface. The fiber knob receptor is a 45 kDa cell surface protein which has potential sites for both glycosylation and phosphorylation. (Bergelson et al., (1997), Science 275:1320-1323). A secondary method of entry for adenovirus is through integrins present on the cell surface. Arginine-Glycine-Aspartic Acid (RGD) sequences of the adenoviral penton base protein bind integrins on the cell surface.

The adenovirus genome can be manipulated such that it encodes and expresses a nucleic acid of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Representative adenoviral vectors derived from the adenovirus strain Ad type 5 d1324 or other strains of adenovirus (e.g., Ad2, Ad3, Ad7 etc.) are known to those skilled in the art.

Recombinant adenoviruses can be advantageous in certain circumstances in that they are not capable of infecting nondividing cells and can be used to infect a wide variety of cell types, including epithelial cells. Furthermore, the virus particle is relatively stable and amenable to purification and concentration, and can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situations where introduced DNA becomes integrated into the host genome (e.g., as occurs with retroviral DNA). Moreover, the carrying capacity of the adenoviral genome for foreign DNA is large relative to other delivery vectors (Haj-Ahmand and Graham (1986) J. Virol. 57:267).

In particular embodiments, the adenovirus genome contains a deletion therein, so that at least one of the adenovirus genomic regions does not encode a functional protein. For example, first-generation adenovirus vectors are typically deleted for the E1 genes and packaged using a cell that expresses the E1 proteins (e.g., 293 cells). The. E3 region is also frequently deleted as well, as there is no need for complementation of this deletion. In addition, deletions in the E4, E2a, protein IX, and fiber protein regions have been described, e.g., by Armentano et al, (1997) J. Virology 71:2408, Gao et al., (1996) J. Virology 70:8934, Dedieu et al., (1997) J. Virology 71;4626, Wang et al., (1997) Gene Therapy 4:393, U.S. Pat. No. 5,882,877 to Gregory et al. (the disclosures of which are incorporated herein in their entirety). Preferably, the deletions are selected to avoid toxicity to the packaging cell. Wang et al., (1997) Gene Therapy 4:393, has described toxicity from constitutive co-expression of the E4 and E1 genes by a packaging cell line. Toxicity can be avoided by regulating expression of the E1 and/or E4 gene products by an inducible, rather than a constitutive, promoter. Combinations of deletions that avoid toxicity or other deleterious effects on the host cell can be routinely selected by those skilled in the art.

As further examples, in particular embodiments, the adenovirus is deleted in the polymerase (pol), preterminal protein (pTP), IVa2 and/or 100K regions (see, e.g., U.S. Pat. No. 6,328,958; PCT publication WO 00/12740; and PCT publication WO 02/098466; Ding et al., (2002) Mol. Ther. 5:436; Hodges et al., J. Virol. 75:5913; Ding et al., (2001) Hum Gene Ther12:955; the disclosures of which are incorporated herein by reference in their entireties for the teachings of how to make and use deleted adenovirus vectors for gene delivery).

The term “deleted” adenovirus as used herein refers to the omission of at least one nucleotide from the indicated region of the adenovirus genome. Deletions can be greater than about 1, 2, 3, 5, 10, 20, 50, 100, 200, or even 500 nucleotides. Deletions in the various regions of the adenovirus genome can be about at least 1%, 5%, 10%, 25%, 50%, 75%, 90%, 95%, 99%, or more of the indicated region. Alternately, the entire region of the adenovirus genome is deleted. Preferably, the deletion will prevent or essentially prevent the expression of a functional protein from that region. In general, larger deletions are preferred as these have the additional advantage that they will increase the carrying capacity of the deleted adenovirus for a heterologous nucleotide sequence of interest. The various regions of the adenovirus genome have been mapped and are understood by those skilled in the art (see, e.g., FIELDS et al., VIROLOGY, volume 2, chapters 67 and 68 (3d ed., Lippincoff-Raven Publishers).

Those skilled in the art will appreciate that typically, with the exception of the E3 genes, any deletions will need to be complemented in order to propagate (replicate and package) additional virus, e.g., by transcomplementation with a packaging cell.

The present invention can also be practiced with “gutted” adenovirus vectors (as that term is understood in the art, see e.g., Lieber et al., (1996) J. Virol. 70:8944-60) in which essentially all of the adenovirus genomic sequences are deleted.

Adeno-associated viruses (AAV) have also been employed as nucleic acid delivery vectors. For a review, see Muzyczka et al. Curr. Topics in Micro. and Immunol. (1992) 158:97-129). MV are parvoviruses and have small icosahedral virions, 18-26 nanometers in diameter and contain a single stranded genomic DNA molecule 4-5 kilobases in size. The viruses contain either the sense or antisense strand of the DNA molecule and either strand is incorporated into the virion. Two open reading frames encode a series of Rep and Cap polypeptides. Rep polypeptides (Rep50, Rep52, Rep68 and Rep78) are involved in replication, rescue and integration of the MV genome, although significant activity can be observed in the absence of all four Rep polypeptides. The Cap proteins (VP1, VP2, VP3) form the virion capsid. Flanking the rep and cap open reading frames at the 5′ and 3′ ends of the genome are 145 basepair inverted terminal repeats (ITRs), the first 125 basepairs of which are capable of forming Y- or T-shaped duplex structures. It has been shown that the ITRs represent the minimal cis sequences required for replication, rescue, packaging and integration of the MV genome. Typically, in recombinant MV vectors (rMV), the entire rep and cap coding regions are excised and replaced with a heterologous nucleic acid of interest.

AAV are among the few viruses that can integrate their DNA into non-dividing cells, and exhibit a high frequency of stable integration into human chromosome 19 (see, for example, Flotte et al. (1992) Am. J. Respir. Cell. Mol. Biol. 7:349-356; Samulski et al., (1989) J Virol. 63:3822-3828; and McLaughlin et al., (1989) J. Virol. 62:1963-1973). A variety of nucleic acids have been introduced into different cell types using AAV vectors (see, for example, Hermonat et al., (1984) Proc. Natl. Acad. Sci. USA 81:6466-6470; Tratschin et al., (1985) Mol. Cell. Biol. 4:2072-2081; Wondisford et al., (1988) Mol. Endocrinol. 2:32-39; Tratschin et al., (1984) J. Virol. 51:611-619; and Flofte et al., (1993) J. Biol. Chem. 268:3781-3790).

A rAAV vector genome will typically comprise the AAV terminal repeat sequences and packaging signal. An “AAV particle” or “rAAV particle” comprises an AAV vector genome or rAAV vector genome, respectively, packaged within an AAV capsid. The rAAV vector itself need not contain AAV genes encoding the capsid and Rep proteins. In particular embodiments of the invention, the rep and/or cap genes are deleted from the AAV genome. In a representative embodiment, the rAAV vector retains only the terminal AAV sequences (ITRs) necessary for integration, excision, replication.

Sources for the AAV capsid genes can include serotypes AAV-1, AAV-2, AAV-3 (including 3a and 3b.), AAV-4, AAV-5, AAV-6, AAV-7, AAV-8, as well as bovine AAV and avian AAV, and any other virus classified by the International Committee on Taxonomy of Viruses (ICTV) as an AAV (see, e.g., BERNARD N. FIELDS et al., VIROLOGY, volume 2, chapter 69 (4th ed., Lippincott-Raven Publishers).

Because of packaging limitations, the total size of the rAAV genome will preferably be less than about 5.2, 5, 4.8, 4.6, 4.5 or 4.2 kb in size.

Any suitable method known in the art can be used to produce AAV vectors expressing the nucleic acids encoding LDH of this invention (see, e.g., U.S. Pat. No. 5,139,941; U.S. Pat. No. 5,858,775; U.S. Pat. No. 6,146,874 for illustrative methods). In one particular method, AAV stocks can be produced by co-transfection of a rep/cap vector encoding AAV packaging functions and the template encoding the AAV vDNA into human cells infected with the helper adenovirus (Samulski et al., (1989) J. Virology 63:3822).

In other particular embodiments, the adenovirus helper virus is a hybrid helper virus that encodes AAV Rep and/or capsid proteins. Hybrid helper Ad/AAV vectors expressing AAV rep and/or cap genes and methods of producing AAV stocks using these reagents are known in the art (see, e.g., U.S. Pat. No. 5,589,377; and U.S. Pat. No. 5,871,982, U.S. Pat. No. 6,251,677; and U.S. Pat. No. 6,387,368). Preferably, the hybrid Ad of the invention expresses the AAV capsid proteins (i.e., VP1, VP2, and VP3). Alternatively, or additionally, the hybrid adenovirus can express one or more of AAV Rep proteins (i.e., Rep40, Rep52, Rep68 and/or Rep78). The AAV sequences can be operatively associated with a tissue-specific or inducible promoter.

The AAV rep and/or cap genes can alternatively be provided by a packaging cell that stably expresses the genes (see, e.g., Gao et al., (1998) Human Gene Therapy 9:2353; Inoue etal., (1998) J. Virol. 72:7024; U.S. Pat. No. 5,837,484; WO 98/27207; U.S. Pat. No. 5,658,785; WO 96/17947).

Another vector for use in the present invention comprises Herpes Simplex Virus (HSV). Herpes simplex virions have an overall diameter of 150 to 200 nm and a genome consisting of one double-stranded DNA molecule that is 120 to 200 kilobases in length. Glycoprotein D (gD) is a structural component of the HSV envelope that mediates virus entry into host cells. The initial interaction of HSV with cell surface heparin sulfate proteoglycans is mediated by another glycoprotein, glycoprotein C (gC) and/or glycoprotein B (gB). This is followed by interaction with one or more of the viral glycoproteins with cellular receptors. It has been shown that glycoprotein D of HSV binds directly to Herpes virus entry mediator (HVEM) of host cells. HVEM is a member of the tumor necrosis factor receptor superfamily (Whitbeck et al., (1997), J. Virol.; 71:6083-6093). Finally, gD, gB and the complex of gH and gL act individually or in combination to trigger pH-independent fusion of the viral envelope with the host cell plasma membrane. The virus itself is transmitted by direct contact and replicates in the skin or mucosal membranes before infecting cells of the nervous system for which HSV has particular tropism. It exhibits both a lytic and a latent function. The lytic cycle results in viral replication and cell death. The latent function allows for the virus to be maintained in the host for an extremely long period of time.

HSV can be modified for the delivery of nucleic acids to cells by producing a vector that exhibits only the latent function for long-term gene maintenance. HSV vectors are useful for nucleic acid delivery because they allow for a large DNA insert of up to or greater than 20 kilobases; they can be produced with extremely high titers; and they have been shown to express nucleic acids for a long period of time in the central nervous system as long as the lytic cycle does not occur.

In other particular embodiments of the present invention, the delivery vector of interest is a retrovirus. Retroviruses normally bind to a virus-specific cell surface receptor, e.g., CD4 (for HIV); CAT (for MLV-E; ecotrodpic Murine leukemic virus E); RAM1/GLVR2 (for murine leukemic virus-A; MLV-A); GLVR1 (for Gibbon Ape leukemia virus (GALV) and Feline leukemia virus B (FeLV-B)). The development of specialized cell lines (termed “packaging cells”) which produce only replication-defective retroviruses has increased the utility of retroviruses for gene therapy, and defective retroviruses are characterized for use in gene transfer for gene therapy purposes (for a review,. see Miller, (1990) Blood 76:271). A replication-defective retrovirus can be packaged into virions which can be used to infect a target cell through the use of a helper virus by standard techniques.

Yet another suitable vector is a poxvirus vector. These viruses are very complex, containing more than 100 proteins, although the detailed structure of the virus is presently unknown. Extracellular forms of the virus have two membranes while intracellular particles only have an inner membrane. The outer surface of the virus is made up of lipids and proteins that surround the biconcave core. Poxviruses are antigenically complex, inducing both specific and cross-reacting antibodies after infection. Poxvirus receptors are not presently known, but it is likely that there exists more than one given the tropism of poxvirus for a wide range of cells. Poxvirus gene expression is well studied due to the interest in using vaccinia virus as a vector for expression of nucleic acids.

In addition to viral transfer methods, such as those illustrated above, non-viral methods can also be employed. Many non-viral methods of nucleic acid transfer rely on normal mechanisms used by mammalian cells for the uptake and intracellular transport of macromolecules. In particular embodiments, non-viral nucleic acid delivery systems rely on endocytic pathways for the uptake of the nucleic acid molecule by the targeted cell. Exemplary nucleic acid delivery systems of this type include liposomal derived systems, poly-lysine conjugates, and artificial viral envelopes.

In particular embodiments, plasmid vectors are used in the practice of the present invention. Naked plasmids can be introduced into muscle cells by injection into the tissue. Expression can extend over many months, although the number of positive cells is typically low (Wolff et al., (1989) Science 247:247). Cationic lipids have been demonstrated to aid in introduction of nucleic acids into some cells in culture (Felgner and Ringold, (1989) Nature 337:387). Injection of cationic lipid plasmid DNA complexes into the circulation of mice has been shown to result in expression of the DNA in lung (Brigham et al., (1989) Am. J. Med. Sci. 298:278). One advantage of plasmid DNA is that it can be introduced into non-replicating cells.

In a representative embodiment, a nucleic acid molecule (e.g., a plasmid) can be entrapped in a lipid particle bearing positive charges on its surface and, optionally, tagged with antibodies against cell surface antigens of the target tissue (Mizuno et al., (1992) No Shinkei Geka 20:547; PCT publication WO 91/06309; Japanese patent application 1047381; and European patent publication EP-A-43075).

Liposomes that consist of amphiphilic cationic molecules are useful non-viral vectors for nucleic acid delivery in vitro and in vivo (reviewed in Crystal, Science 270: 404-410 (1995); Blaese et al., Cancer Gene Ther. 2: 291-297 (1995); Behr et al., Bioconjugate Chem. 5: 382-389 (1994); Remy et al., Bioconjugate Chem. 5: 647-654 (1994); and Gao et al., Gene Therapy 2: 710-722 (1995)). The positively charged liposomes are believed to complex with negatively charged nucleic acids via electrostatic interactions to form lipid:nucleic acid complexes. The lipid:nucleic acid complexes have several advantages as nucleic acid transfer vectors. Unlike viral vectors, the lipid:nucleic acid complexes can be used to transfer expression cassettes of essentially unlimited size. Since the complexes lack proteins, they can evoke fewer immunogenic and inflammatory responses. Moreover, they cannot replicate or recombine to form an infectious agent and have low integration frequency. A number of publications have demonstrated that amphiphilic cationic lipids can mediate nucleic acid delivery in vivo and in vitro (Felgner et al., Proc. Natl. Acad. Sci. USA 84: 7413-17 (1987); Loeffler et al., Methods in Enzymology 217: 599-618 (1993); Felgner et al., J. Biol. Chem. 269: 2550-2561 (1994)).

Several groups have reported the use of amphiphilic cationic lipid:nucleic acid complexes for in vivo transfection both in animals and in humans (reviewed in Gao et al., Gene Therapy 2: 710-722 (1995); Zhu et al., Science 261: 209-211 (1993); and Thierry et al., Proc. Natl. Acad. Sci. USA 92: 9742-9746 (1995)). U.S. Pat. No. 6,410,049 describes a method of preparing cationic lipid:nucleic acid complexes that have a prolonged shelf life.

Having now described the invention, the same will be illustrated with reference to certain examples, which are included herein for illustration purposes only, and which are not intended to be limiting of the invention.

### EXAMPLE 1

### Cell Culture

The cell line 832/13 was created from INS-1 insulinoma β-cells as described earlier (Hohmeier et al. (2000) Diabetes 49:424-430). Cells were cultured in RPMI-1640 medium containing 11 mM glucose supplemented with 10% fetal bovine serum, 10 mM HEPES, 2 mM glutamine, 1 mM sodium pyruvate and 50 mM β-mercaptoethanol. Media was routinely changed every second day.

### EXAMPLE 2

### Recombinant Adenovirus

A cDNA clone containing the coding sequence of the rat cytoplasmic LDH_{A} isoform surrounded by a EcoRI/BamHI linker (underlined in primers) was made by PCR amplification of rat liver cDNA using the following primers 5′-GGA ATT CGT GTG CTG GAG CCA CTG T-3′ (sense, SEQ ID NO:12), and 5′-CGC GGA TCC TGT AGA ACA TTT TAT GCA C-3′ (antisense, SEQ ID NO:13), and subsequently ligated into the EcoRI/BamHl sites of pAC CMV.pLpA. After sequencing, pAcCMV.pLpA/LDH_{A} was cotransfected with pJM17 into low passage HK-293 cells for virus propagation as described earlier (Becker et al. (1994) Methods Cell Biol. 43(Pt A):161-89; Becker et al., (1994) J. Biol. Chem.269:21234-8).

### EXAMPLE 3

### Cloning of Mitochondrial Form of LDH_{A}

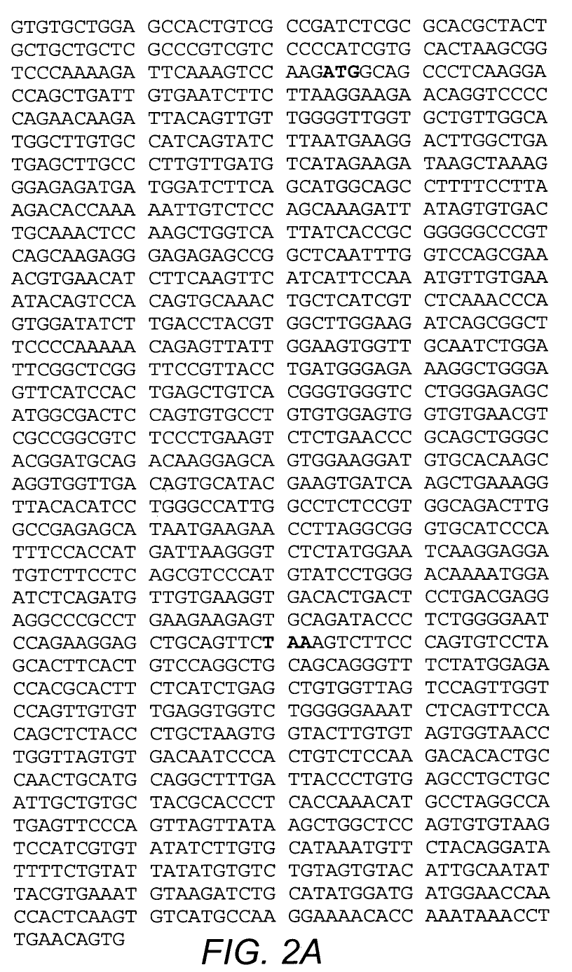

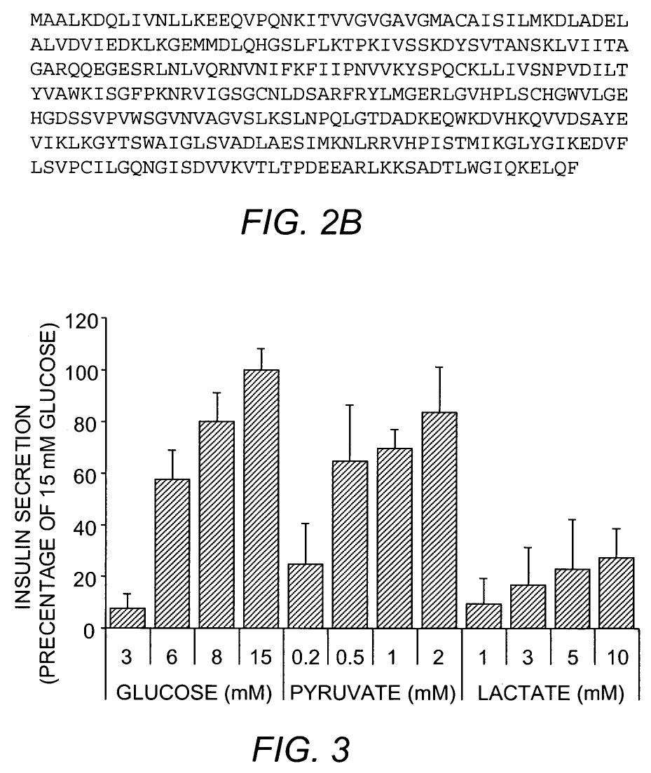

DNase treated RNA purified from rat liver by RNeasy kit (Qiagen) was utilized as template for cloning of the rat mitochondrial form of LDH_{A} using SuperScript one-step RT-PCR with platinum Taq (Invitrogen) and the following primers 5′-CGC TCT ACT TGC TGT AGG-3′ (sense 5′LDHexonmit, SEQ ID NO:14) and 5′-GCC TGG ACA GTG MG TGC TAG G-3′ (antisense 3′cloning SEQ ID NO:15). RT-PCR reaction was performed according to the manufacturer's recommendations with the following cycling conditions: cDNA synthesis and pre-denaturation: 50° C. for 30 min, 94° C. for 2 min. PCR amplification: 2 cycles (94° C. for 15 s, 56° C. for 30 s, 68° C. for 90 s), 2 cycles (94° C. for 15 s, 54° C. for 30 s, 68° C. for 90 s), 30 cycles (94° C. for 15 s, 52° C. 30 s, 68° C. for 90 s). Final extension 72° C. for 5 min. 1 μl PCR reaction was then used as template for an additional PCR reaction with the following cycling conditions 94° C. for 2 min, 30 cycles (94° C. for 15 s, 53° C. for 30 s, 72° C. for 90 s) and 72° C. for 5 min. The resulting PCR product of 1.2 kb was cloned into the TOPO® TA blunt vector (Invitrogen) and sequenced (FIG. 2A; SEQ ID NO:3).

### EXAMPLE 4

### Recombinant mitLDH_{A} Adenovirus

With the purpose of constructing a mitLDH_{A} adenovirus, DNAse treated RNA purified from rat liver by RNeasy kit (Qiagen) was utilized as template for cloning of the mitochondrial form of LDH using SuperScript one-step RT-PCR with platinum Taq (Invitrogen) and the following primers 5′-AAC CGT GTA AGA GGA GGG ACC ATC-3′ (sense, SEQ ID NO:16) and 5′-TGG ACC AAC TGG ACT MC CAC AGC-3′ (antisense, SEQ ID NO:17). Cycling conditions: cDNA synthesis and pre-denaturation: 53° C. for 30 min, 94° C. for 2 min. PCR amplification: 2 cycles (94° C. for 15 s, 63° C. for 30 s, 68° C. for 90 s), 2 cycles (94° C. for 15 s, 61° C. for 30 s, 68° C. for 90 s), 30 cycles (94° C. for 15 s, 59° C. for 30 s, 68° C. for 90 s). Final extension 72° C. for 5 min. The resulting PCR product of 1.2 kb was cloned into the TOPO® TA blunt vector (Invitrogen). Subsequently, the insert was cut out with EcoRI and ligated into pAC CMV.pLpA and resulting clones were tested for orientation by restrictions digest and sequencing. Virus were made as described above.

### EXAMPLE 5

### Virus Transduction

832/13 cells were split to a cell density of 20-30% confluence in 12 well plates. At a cell density of 90%, cells were exposed to virus by adding crude extract directly to the growth media. After 16 hours of transduction, virus containing media was removed and the cells were cultured in fresh media in additional 24 hours before secretion assays.

### EXAMPLE 6

### Secretion Assays

Insulin secretion was assayed in HEPES buffered saline solution (HBSS) (114 mM NaCl, 4.7 mM KCl,1.2 mM KH₂PO₄, 1.16 mM MgSO₄, 20 mM HEPES, 2.5 mM CaCl₂, 25.5 mM NaHCO₃, pH 7.2 containing 0.2% bovine serum albumin (essentially fatty acid free). Cells were washed in 1 ml of HBSS, preincubated in 1.5 ml of HBSS for 2 hour prior to a 2 hour incubation in the presence of secretagogues as indicated in the figures. The insulin levels were determined by radioimmunoassay with the Coat-A-Count kit (ICN Pharmaceuticals, Costa Mesa, Calif.) and lactate output was determined with a Lactate Reagent kit (Sigma, St. Louis, Mo.) according to protocols outlined by the manufacturer.

### EXAMPLE 7

### LDH Activity Assay and Protein Levels

832/13 cells in 12 well plates were washed in 1 ml Phosphate Buffered Saline (PBS, 10 mM Potassium phosphate, 120 mM NaCl, 2.7 mM KCl, pH 7.4) and subsequently lysed in 50 ml PBS containing 0.1% Triton X100. For LDH activity, 0.5-10 ml of extract was incubated in a buffer containing 50 mM potassium phosphate, 0.63 mM sodium pyruvate, pH 7.5, and the enzyme reaction is initiated by adding NADH to a final concentration of 0.18 mM, and activity was measured by the decrease in absorbance at 340 nm at RT. Protein levels were determined using a Bio-Rad protein assay (Bio-Rad, Hercules, Calif.) according to protocols outlined by the manufacturer.

### EXAMPLE 8

### Glucose Usage

In order to determine glycolytic flux, the insulin secretion assay was performed in the presence [³H] glucose (specific activity 2×10⁴ cpm/ml). After 2 hours incubation, the HBSS was collected, proteins were removed by centrifugation (2 min, 12000 g) after adding trichloric acid to a final concentration of 4%. Microtubes (1.5 ml without cap) containing the supernatants were placed in tightly closed scintillation tubes containing 500 μl H₂O and placed at 50° C. overnight. After cooling to RT, glucose usage was determined from the level of ³H₂O released to the scintillation tubes.

### EXAMPLE 9

### Effect of LDH_{A} on Glucose and Pyruvate Stimulation of IS

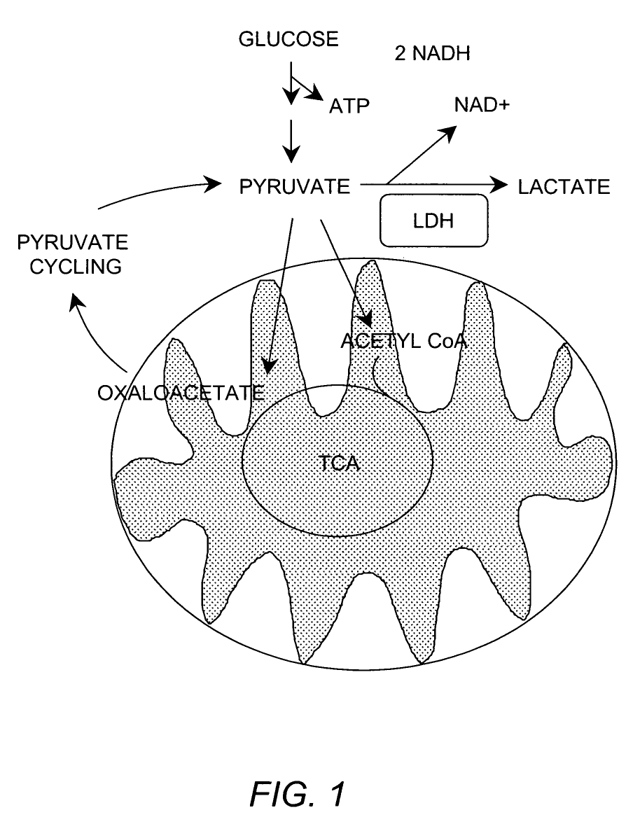

LDH catalyzes the conversion of pyruvate to lactate (FIG. 1). Based on earlier observations showing a direct correlation between pyruvate cycling and insulin secretion, it was hypothesized that the effect of overexpression of LDH_{A}—if any—would be impaired IS response consistent with earlier observations (Alcazar et al. (2000) Biochem. J. 352:373-380; Ishihara et al. (1999) J. Clin. Invest. 104:1621-1629; Ainscow et al. (2000) Diabetes 49:1149-1155). Lactate has been observed to be a poor secretagogue compared to pyruvate and glucose of insulin (FIG. 3).

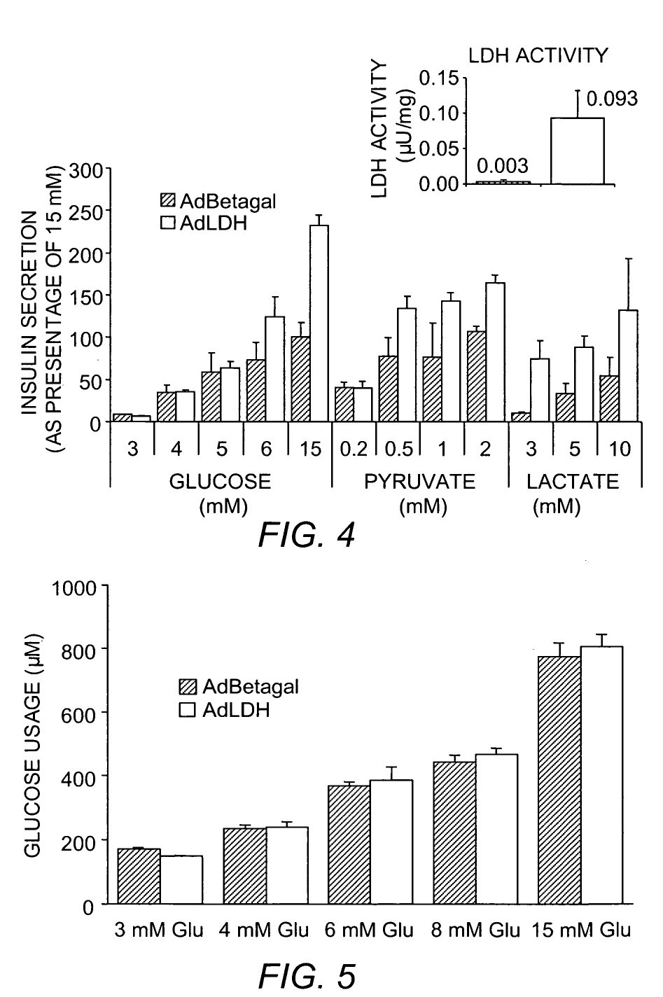

Surprisingly, overexpression of rat LDH_{A} in our 832/13 cell model potentiates IS in response to glucose and pyruvate (FIG. 4) without affecting glycolytic flux with glucose as secretagogue (FIG. 5). In the absence of the overexpression of LDH_{A}, lactate is a weak stimulator of IS. However, in 832/13 cells treated with adenovirus expressing rat LDH_{A} (AdLDH), lactate stimulation of IS was potentiated (FIG. 4). In cells overexpressing LDH_{A}, lactate is as effective as glucose or pyruvate as an insulin secretagogue.

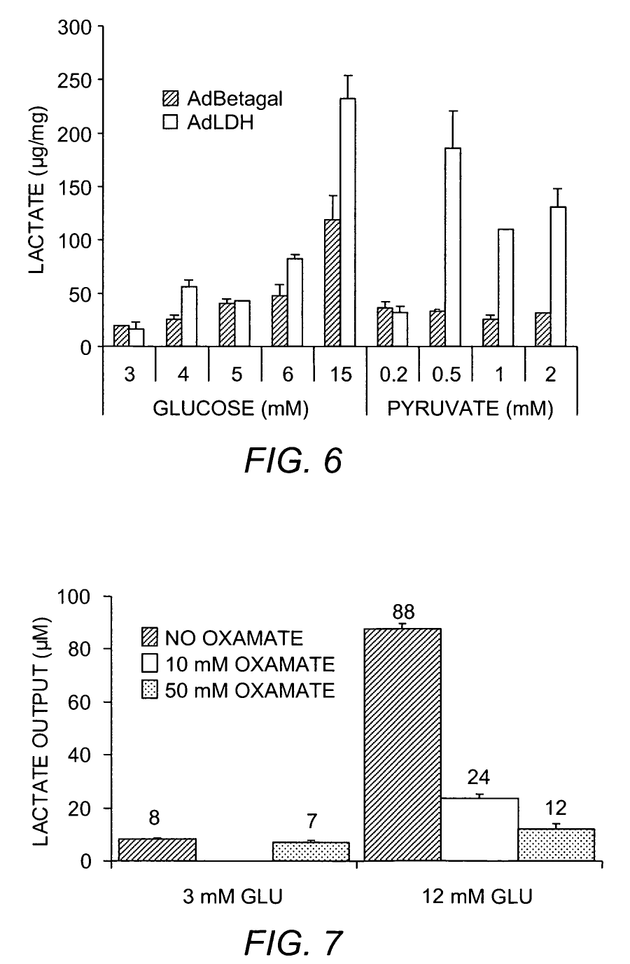

When lactate production was examined, it was observed that rat LDH_{A} overexpression increases lactate output in response to both glucose and pyruvate (FIG. 6), showing that the LDH driven process occurs in the direction of lactate. This indicates that lactate itself might be involved in regulation of IS. However, in the absence of LDH_{A} overexpression, lactate is only a weak stimulator of IS (FIG. 3).

### EXAMPLE 10

### Effect of Oxamate on Pyruvate Cycling and IS

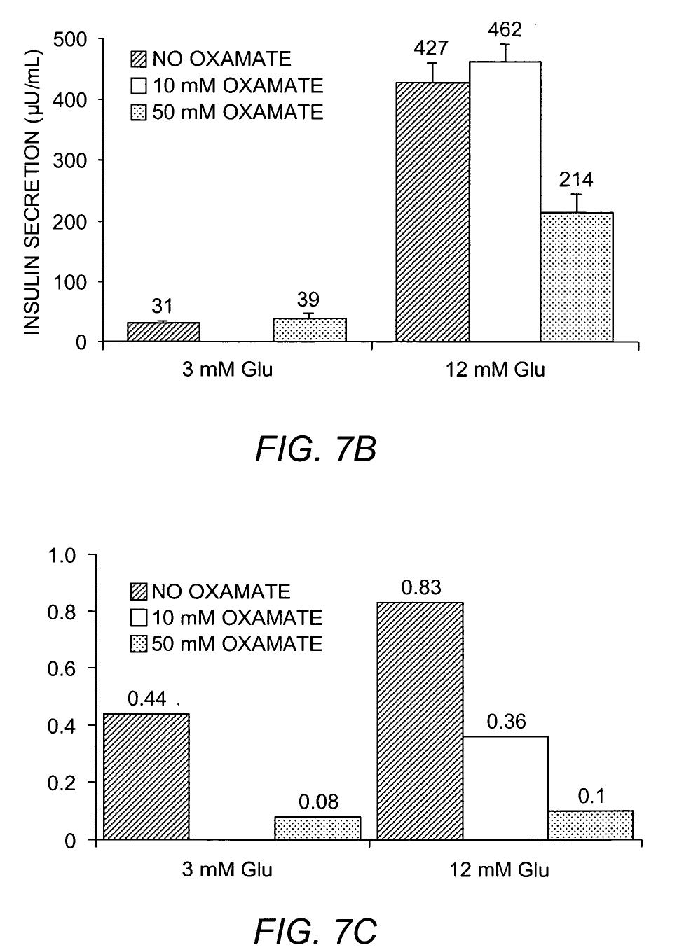

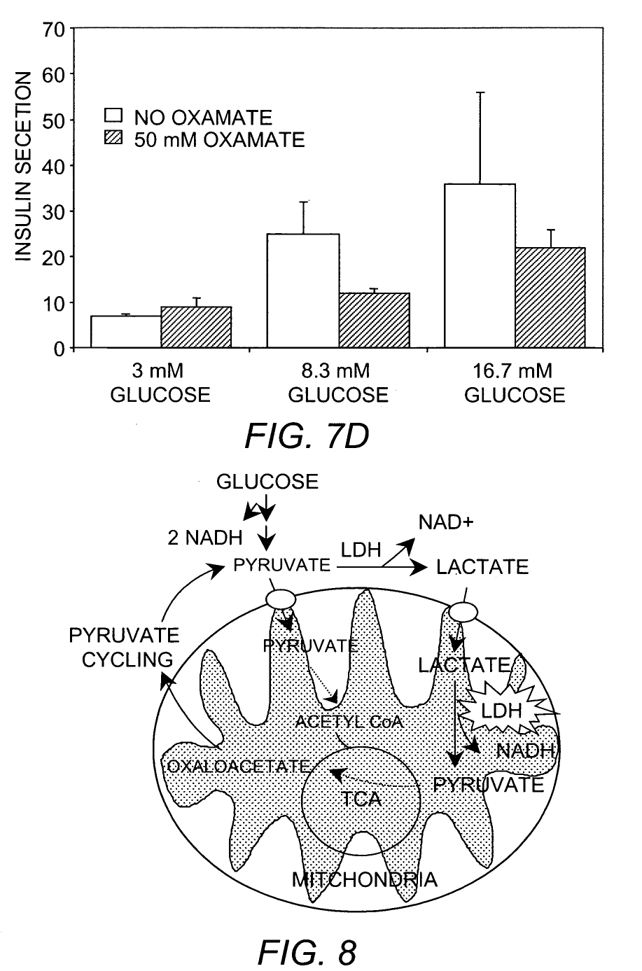

¹³C NMR analyses demonstrated a correlation between lactate output, pyruvate cycling and insulin secretion in the presence and absence of the LDH inhibitor, oxamate in 823/13 cells (FIGS. 7 A-C). Oxamate inhibition of glucose-stimulated insulin secretion was also observed in pancreatic islet β-cells (FIG. 7D).

### EXAMPLE 11

### Mitochondrial LDH_{A} is Implicated in Fuel-Stimulated IS

The results shown in Example 10 are consistent with the regulation of fuel-stimulated IS involving a mitochondrial form of LDH following the scheme shown in FIG. 8. According to this model, both lactate and pyruvate are transported into the mitochondria via the monocarboxylate transporter. Inside the mitochondria, lactate is then converted to pyruvate via the mitochondrial LDH. In our model, both entry pathways must be active in order to obtain IS. With lactate as secretagogue, the flux though the pyruvate entry path is limiting due to low level of LDH. However, when LDH is overexpressed, the flux though the pyruvate entry pathway increases which in turn increases IS. On the other hand, with pyruvate or glucose as secretagogues, the lactate entry way is limiting for IS and an increase in LDH expression will increase the flux thereby resulting in a potentiation of IS. In addition, this scheme can also explain our earlier observation of two distinct pools of pyruvate present in beta cells (Lu et al., (2000) Proc. Natl. Acad. Sci. USA 99:2708).

### EXAMPLE 12

### Correlation of Pyruvate Cycling with Lactate Production in Glycerol Kinase Producing Cells

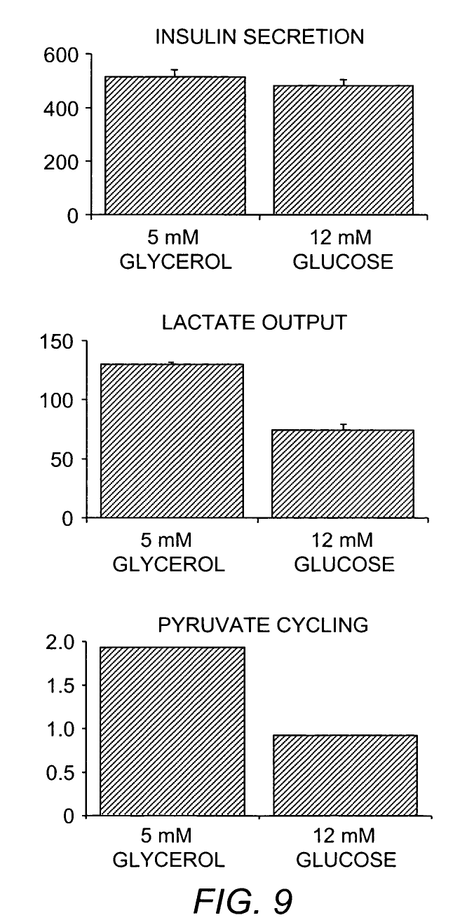

There is minimal expression of glycerol kinase in β-cell s, thus glycerol is not converted to glucose in β-cells, and does not stimulate insulin secretion. FIG. 9 shows ¹³C studies that indicate lactate output correlates with pyruvate cycling in glycerol kinase overexpressing 832/13 cells. Glucose and glycerol stimulate insulin secretion to the same extent in glycerol kinase overexpressing cells. In contrast, pyruvate cycling and lactate output are higher in response to glycerol than to glucose.

### EXAMPLE 13

### Organization of the LDH_{A} Gene and the Identification of an Alternative Exon

The structure of the mouse LDH_{A} gene (GenBank Accession No. Y00309) is shown in FIG. 10. The gene spans 12.9 kb and the cytosolic LDH_{A} mRNA contains 8 exons. Analysis of the complete mouse LDH_{A} gene sequence revealed an alternative exon (SEQ ID NO:18), when spliced to the 5′ end of exon 2 through alternative exon usage will give rise to a mRNA containing an amino-terminal extension of the LDH protein sequence of 29 amino acids (FIG. 7, SEQ ID NO:20). FIG. 11 depicts the leader sequences for mouse (SEQ ID NO:20), rat (SEQ ID NO:21), and human (SEQ ID NO:22) mit-LDH_{A}. Conservation of the amino acid sequence of this 29 amino acid leader peptide sequence was revealed and a consensus sequence (SEQ ID NO:23) from these sequences is shown (FIG. 11).

### EXAMPLE 14

### Cloning of the Mitochondrial Form of LDH_{A} from Rat

Utilizing the PCR primers and protocol outlined in Example 3, a cDNA clone containing the mitochondrial form of rat LDH_{A} (mitLDH_{A}) was isolated. The sequence of this cDNA clone (SEQ ID NO:3) is shown in FIG. 12A. The translation of the open reading frame from nucleotides 101-1186 giving rise to a protein of 361 amino acids (FIG. 12B, SEQ ID NO:4) having a 29 amino acid leader sequence added to the cytoplasmic LDH_{A} polypeptide sequence (FIG. 2B, SEQ ID NO:2). The presence of this transcript in pancreatic 832/13 cells is confirmed by RT-PCR.

### EXAMPLE 15

### Comparisons of Mitochondrial Forms of LDH_{A} from Rat, Mouse, and Human

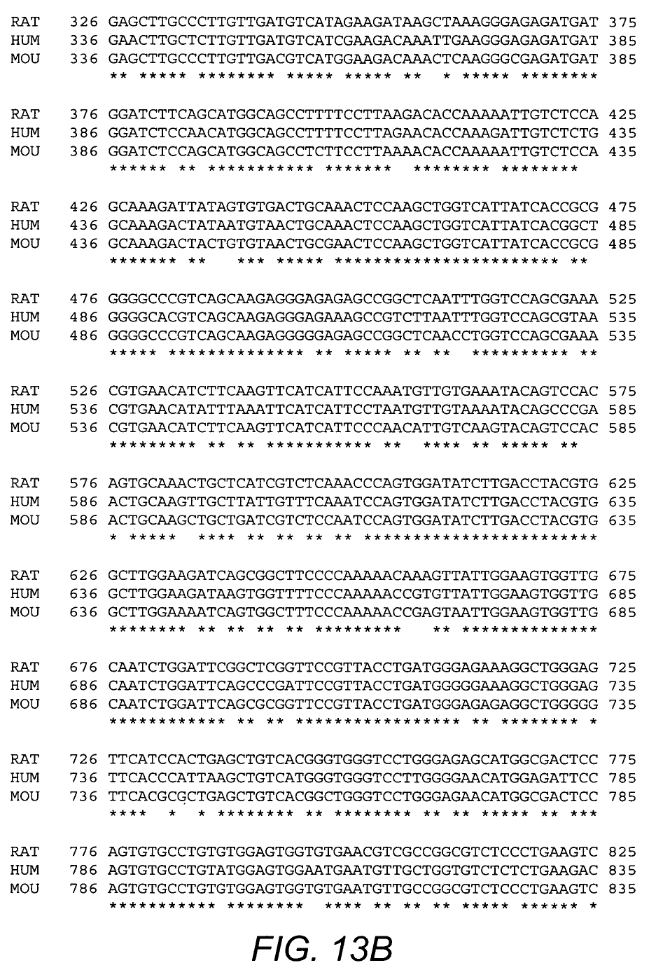

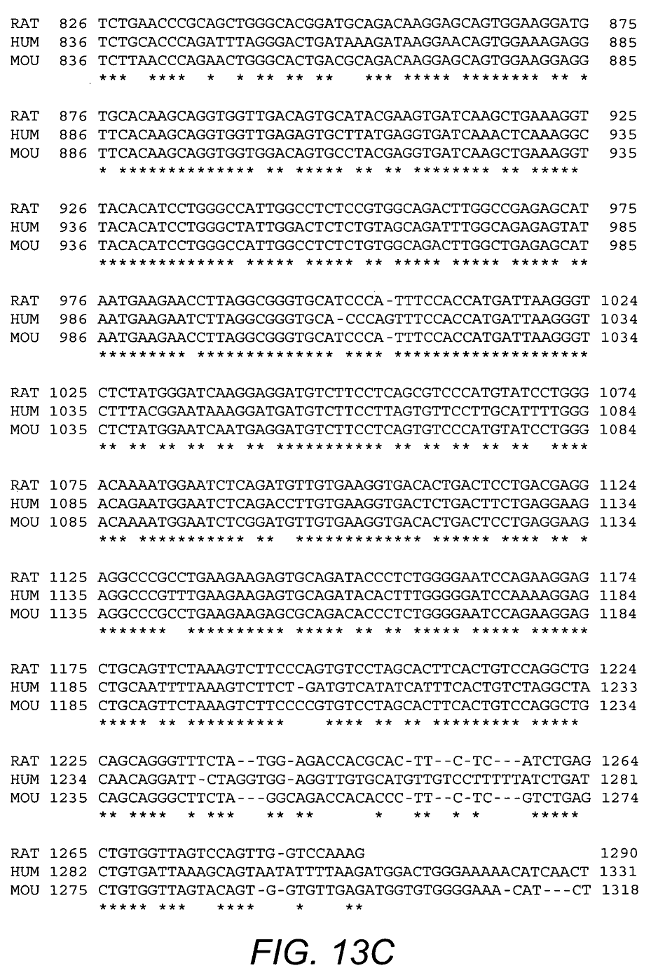

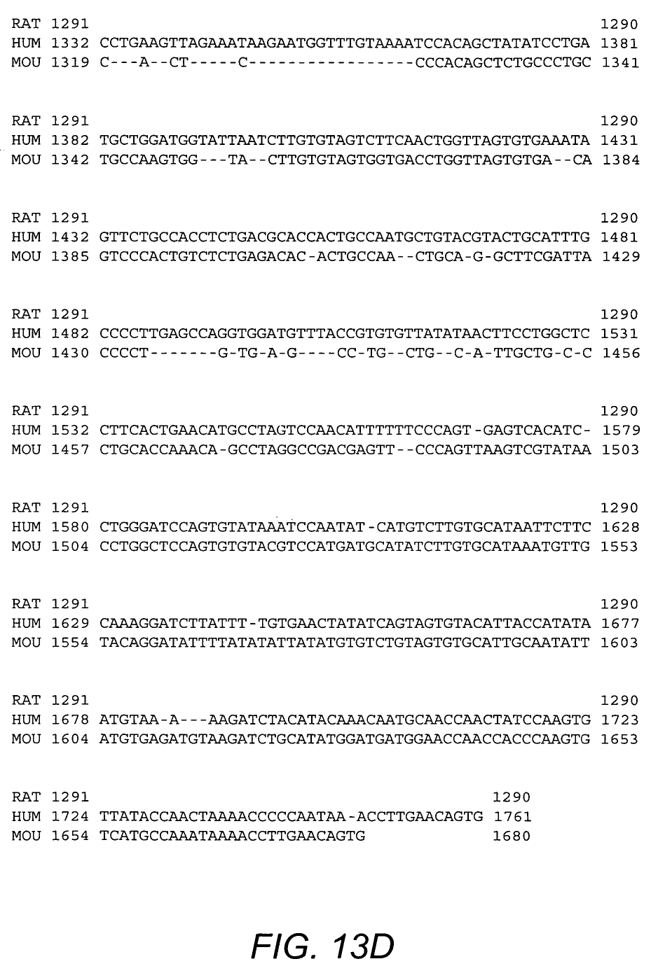

The mouse mRNA (SEQ ID NO:24), the translation of the open reading frame from nucleotides 111-1193 for the mouse mitochondrial LDH (SEQ ID NO:25) are shown in FIGS. 12C and 12D respectively. The human mRNA (SEQ ID NO:26), the translation of the open reading frame from nucleotides 111-1193 for the human mitochondrial LDH (SEQ ID NO:27) are shown in FIGS. 12E and 12F respectively. An alignment of the mouse and human mRNAs with the sequence from the rat cDNA (SEQ ID NO:3) for mitochondrial LDH is shown in FIGS. 13 A-D.

### EXAMPLE 16

### Effect of the Mitochondrial Form of LDH_{A} on Fuel Mediated IS

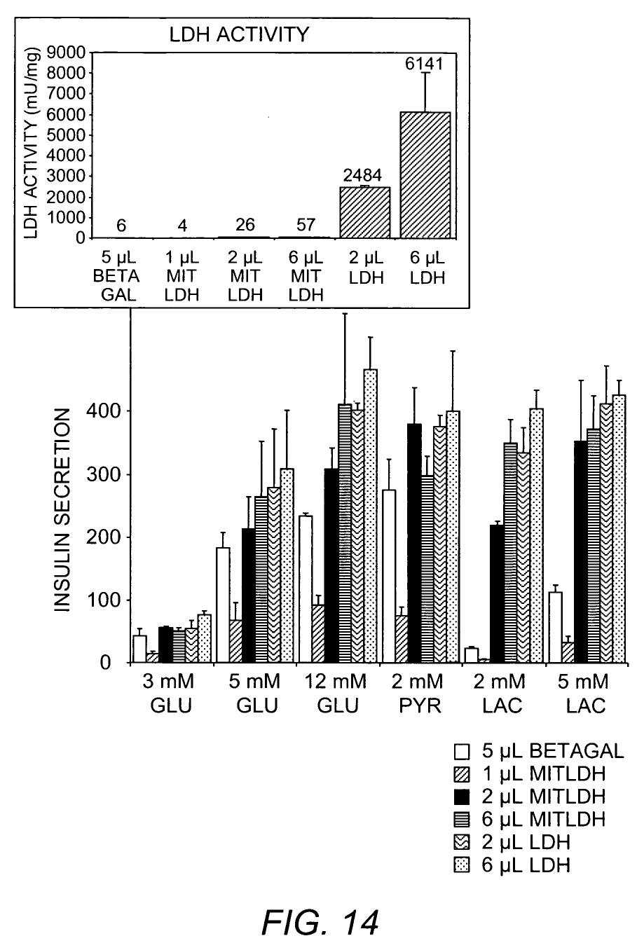

Example 9 describes the effects of overexpression of LDH_{A} on glucose, pyruvate, and lactate mediated IS. The effects of rat mitLDH_{A} on fuel mediated IS were examined and compared with rat cytosdlic LDH_{A}, the results of which are shown in FIG. 14. These results show that mitLDH_{A} is capable of potentiating fuel-mediated IS in a similar manner to rat LDH_{A}, and that the magnitude of the mit-LDH_{A} response occurs at lower enzyme activity this is observed with rat LDH_{A}.

### EXAMPLE 17

### Mass Spectroscopy Based Metabolic Profiling

A mass-spectroscopy (MS) based metabolic profiling approach has been used to understand key differences between robustly glucose responsive and poorly glucose responsive INS-1-derived cell lines. These investigations have involved analysis of glucose-derived metabolites via gas chromatography (GC)/MS. In a-comparison of a robustly glucose responsive cell line 832/13 with a poorly glucose responsive cell line 832/2, new evidence has been found for a role of lactate and pyruvate in glucose stimulated insulin secretion. The analysis involved stimulation of 832/13 or 832/2 cells with 12 mM glucose for 15 minutes, followed by collection of the cellular media and preparation of cell lysates. Focusing on lactate and pyruvate (other intermediates also show interesting changes, and are the subject of ongoing investigations), Table 2 shows that the ratio of lacate_{lysate}: lactate_{media} and pyruvate_{lysate}: pyruvate_{media} is approximately 10-fold higher in 832/13 cells than in 832/2 cells. In other words, the more glucose responsive cell line retains a much higher percentage of lactate and pyruvate within the cell than the less glucose responsive line. One possible explanation for this result is lesser activity of monocarboxylic acid transporters in the former cells than in the latter. This finding suggests that inhibition of monocarboxylic acid transport, can enhance glucose stimulated insulin secretion in 832/2 cells and other cells that lack a robust glucose stimulated insulin secretory response.

TABLE 2
Lactate and pyruvate ratios in cell lysates versus media following
15 minutes of stimulation with 12 mM glucose.

| | Lacate_{lysate}:Lactate_{media} | Pyruvate_{lysate}:Pyruvate_{media} |
|---|---|---|
| 832/13 Cells | 75 | 10 |
| 832/2 Cells | 8.5 | 1.2 |

### EXAMPLE 18

### RNAi Inhibition of Cytosolic LDH Activity

As shown in the previous Examples, the LDH inhibitor oxamate potently inhibits glucose stimulated insulin secretion from 832/13 cells and normal rat islets. Investigations are carried out to provide independent evidence that glucose stimulated insulin secretion is impaired by inhibition of the cytosolic form of LDH. To this end, an RNAi construct specific for cytosolic LDH is delivered to 832/13 cells and normal rat islets. The effects on cytosolic LDH activity and glucose stimulated insulin secretion are assessed.

The foregoing is illustrative of the present invention, and is not to be construed as limiting thereof. The invention is defined by the following claims, with equivalents of the claims to be included therein.

1. An isolated cell comprising an isolated nucleic acid comprising a nucleotide sequence encoding a mitochondrial lactate dehydrogenase (LDH).

2. The cell of claim 1, wherein the cell is an endocrine cell.

3. The cell of claim 1, wherein the cell is a secretory cell capable of forming secretory granules.

4. The cell of claim 1, wherein the cell secretes insulin in response to an elevation in extracellular glucose concentration.

5. The cell of claim 1, wherein the cell is an islet β-cell.

6. The cell of claim 1, wherein the cell is an insulinoma cell.

7. The cell of claim 6, wherein the cell is selected from the group consisting of a β-TC cell, a RIN cell, a HIT cell, a MIN6 cell, a MSL-G2 cell, a INS-1 cell, and a 832/13 cell.

8. The cell of claim 1, wherein said nucleotide-sequence is selected from the group consisting of:
(a) a nucleotide sequence selected from the group consisting of the nucleotide sequence of SEQ ID NO:1, SEQ ID NO:3, SEQ ID NO:24 and SEQ ID NO:26;
(b) a nucleotide sequence that hybridizes to a nucleotide sequence selected from the group consisting of the nucleotide sequence of SEQ ID NO:1, SEQ ID NO:3, SEQ ID NO:24 and SEQ ID NO:26 or its complementary nucleotide sequence under stringent conditions, wherein said nucleotide sequence encodes a functional LDH_{A}; and
(c) a nucleotide sequence encoding an amino acid sequence encoded by the nucleotide sequences of (a) or (b), but which has a different nucleotide sequence than the nucleotide sequences of (a) or (b) due to the degeneracy of the genetic code or the presence of non-translated nucleotide sequences.

9. The cell of claim 8, wherein said nucleotide sequence consists essentially of a nucleotide sequence selected from the group consisting of the nucleotide sequence of SEQ ID NO:3, SEQ ID NO:24 and SEQ ID NO:26.

10. The cell of claim 1, wherein said nucleotide sequence encodes an amino acid sequence having at least about 70% amino acid sequence similarity to an amino acid sequence selected from the group consisting of SEQ ID NO:4, SEQ ID NO:25 and SEQ ID NO:27 or a functional fragment of any of the foregoing.

11. The cell of claim 1, wherein said nucleotide sequence encodes an amino acid sequence comprising an amino acid sequence selected from the group consisting of SEQ ID NO:4, SEQ ID NO:25 and SEQ ID NO:27 or a functional fragment thereof.

12. An isolated cell comprising an isolated nucleic acid comprising a nucleotide sequence encoding a lactate dehydrogenase (LDH), wherein fuel-stimulated insulin secretion is enhanced in the cell.

13. The cell of claim 12, wherein glucose-stimulated insulin secretion is enhanced in the cell.

14. The cell of claim 12, wherein the cell is a secretory cell capable of forming secretory granules.

15. The cell of claim 12, wherein the cell is an endocrine cell.

16. The cell of claim 12, wherein the cell is an islet β-cell.

17. The cell of claim 12, wherein the cell is an insulinoma cell.

18. The cell of claim 17, wherein the cell is selected from the group consisting of a β-TC cell, a RIN cell, a HIT cell, a MIN6 cell, a MSL-G2 cell, an INS-1 cell, and a 832/13 cell.

19. The cell of claim 12, wherein the cell is derived from cells selected from the group consisting of pituitary cells, adrenal cells, and thyroid cells.

20. The cell of claim 19, wherein the cell is derived from AtT-20 cells.

21. The cell of claim 19, wherein the cell is derived from GH-1 or GH-3 cells.

22. The cell of claim 12, Wherein said nucleotide sequence encodes a mitochondrial LDH.

23. The cell of claim 12, wherein said nucleotide sequence encodes a cytoplasmic LDH.

24. The cell of claim 12, wherein said nucleotide sequence encodes a LDH_{A}.

25. A pharmaceutical composition comprising the cell of claim 1 in a pharmaceutically acceptable carrier.

26. A pharmaceutical composition comprising the cell of claim 12 in a pharmaceutically acceptable carrier.

27. A method of providing fuel-stimulated insulin secreting capability to a mammalian subject, comprising:
implanting into a mammalian subject a therapeutically effective amount of a population of cells according to claim 12.

28. The method of claim 27, wherein the cell population is positioned in a selectively permeable membrane.

29. The method of claim 27, wherein the cell population is implanted intraperitoneally or subcutaneously.

30. The method of claim 27, wherein the cell population is implanted within a selectively permeable device that is connected to the vasculature of the mammalian subject.

31. The method of claim 30, wherein the cell population is positioned in a tubular semipermeable membrane positioned within a protective housing.

32. The method of claim 31, wherein each end of said tubular membrane is attached to an arterial graft that extends beyond said housing and joins the device to the vasculature as an arteriovenous shunt.

33. The method of claim 27, wherein the cell population is positioned into a selectively permeable membrane within an implantable device.

34. The method of claim 27, wherein the selectively permeable membrane is a biocompatible coating.

35. The method of claim 34, wherein the cell population is encapsulated by the biocompatible coating.

36. The method of claim 34, wherein the biocompatible coating is a semipermeable capsule.

37. The method of claim 36, wherein the cell population is microencapsulated.

38. The method of claim 36, wherein the cell population is encapsulated in a hydrogel coating.

39. The method of claim 36, wherein the cell population is encapsulated in an alginate coating.

40. The method of claim 27, wherein the cell population is fiber seeded into a semipermeable fiber.

41. The method of claim 27, wherein about 1,000 to about 10,000 cells are encapsulated within a semipermeable capsule or semipermeable fiber.

42. The method of claim 27, wherein the mammalian subject is a diabetic subject.

43. A device comprising a population of cells according to claim 12.

44. The device of claim 43, wherein the cell population is positioned into a selectively permeable membrane within the device.

45. The device of claim 43, wherein the device is an implantable device.

46. The device of claim 43, wherein the cell population is positioned in a tubular semipermeable membrane positioned within a protective housing.

47. The device of claim 46, wherein each end of said tubular membrane is attached to an arterial graft that extends beyond said housing and joins the device to a vascular system as an arteriovenous shunt.

48. The device of claim 43, wherein the selectively permeable membrane is a biocompatible coating.

49. The device of claim 48, wherein the cell population is encapsulated by the biocompatible coating.

50. The device of claim 48, wherein the biocompatible coating is a semipermeable capsule.

51. The device of claim 50, wherein the cell population is microencapsulated.

52. The device of claim 50, wherein the cell population is encapsulated in a hydrogel coating.

53. The device of claim 50, wherein the cell population is encapsulated in an alginate coating.

54. The device of claim 43, wherein the cell population is fiber seeded into a semipermeable fiber.

55. The device of claim 43, wherein about 1,000 to about 10,000 cells are encapsulated within a semipermeable capsule or semipermeable fiber.
